(19) United States
(12) Patent Application Publication
(10) Pub. No.: US 2019/0307692 A1
(43) Pub. Date: Oct. 10, 2019

(54) NANOLIPOPROTEIN PARTICLES AND RELATED COMPOSITIONS METHODS AND SYSTEMS FOR IMPROVED DRUG LOADING

(71) Applicants: Lawrence Livermore National Security, LLC, Livermore, CA (US); The Regents Of The University of California, Oakland, CA (US)

(72) Inventors: Craig D. BLANCHETTE, CONCORD, CA (US); Nicholas FISCHER, LIVERMORE, CA (US); Sean Fitzpatrick GILMORE, OAKLAND, CA (US); Paul HENDERSON, DUBLIN, CA (US)

(21) Appl. No.: 16/082,924

(22) PCT Filed: Mar. 3, 2017
(86) PCT No.: PCT/US2017/020827

§ 371 Date: Sep. 6, 2018

Related U.S. Application Data
(60) Provisional application No. 62/304,852, filed on Mar. 7, 2016.

(51) Int. Cl. A61K 9/127; A61K 31/337

(52) U.S. Cl. CPC A61K 9/1275; A61K 9/1277; A61K 31/337

(57) ABSTRACT

Nanolipoprotein particles comprising at least a scaffold protein component and a membrane lipid component and related compositions, methods and systems are described, in which the membrane lipid component comprises at least one or more membrane forming lipids and one or more lysoplipids.

### CROSS REFERENCE TO RELATED APPLICATIONS

The present application claims priority to U.S. Provisional Application No. 62/304,852, entitled “Nanolipoprotein Particles and Related Compositions Methods and Systems for Improved Drug Loading” filed on Mar. 7, 2016, with docket number IL-13023, the disclosure of which is incorporated by reference in its entirety.

### STATEMENT OF GOVERNMENT GRANT

The invention was made with Government support under Contract No. DE-AC52-07NA27344 between the U.S. Department of Energy and Lawrence Livermore National Security, LLC, for the operation of Lawrence Livermore National Security. The Government may have certain rights to the invention.

### FIELD

The present disclosure relates to nanolipoprotein particles (NLPs) and, in particular, nanolipoprotein particles incorporated with hydrophobic lipid compounds and related compositions methods and systems.

### BACKGROUND

Nanolipoprotein particles are nanometer-sized particles usually comprised of an amphipathic lipid bilayer and an apolipoprotein. NLPs have been used for various biotechnology applications, such as membrane protein stabilization/solubilization, drug delivery, and in particular vaccine delivery, and diagnostic imaging.

In some instances, NLPs can self-assemble under appropriate conditions into nano-scale amphipathic apolipoprotein-stabilized lipid bilayer particles possibly comprising additional molecules, such as one or more integral membrane proteins or other proteins and molecules attached to the amphipathic component of the NLP. The self-assembled particles are typically formed by an apolipoprotein encircling a nanometer scale lipid bilayer defining a nanolipoprotein particle.

Despite the advancement of this technology, providing NLPs including desired functionalities and/or with a desired stability can be challenging.

### SUMMARY

Provided herein, are nanolipoprotein particles, and related compositions, methods and systems, which comprise one or more membrane forming lipids, one or more lysolipid and a scaffold protein. In several embodiments, nanolipoprotein particles herein described can show an increased loading capacity as a carrier for a hydrophobically anchorable target molecule, compared to nanolipoprotein particles assembled in the absence of the lysolipids.

According to a first aspect, a nanolipoprotein particle is described. The nanolipoprotein particle comprises one or more membrane forming lipids, one or more lysolipids and one or more scaffold proteins, the membrane forming lipid and the lysolipids arranged in a membrane lipid bilayer stabilized by the scaffold protein.

According to a second aspect, a nanolipoprotein particle is described. The nanolipoprotein particle comprises a lipid bilayer confined in a discoidal configuration by a scaffold protein, the lipid bilayer comprising one or more lysolipids and one or more membrane forming lipids.

According to a third aspect, a nanolipoprotein particle comprising a hydrophobically anchorable target molecule is described. The nanolipoprotein particle comprises a membrane-forming lipid, one or more lysolipids, the hydrophobically anchorable target molecule and a scaffold protein. In the nanolipoprotein particle, the membrane forming lipid and the one or more lysolipids lipid are arranged in a lipid bilayer stabilized by the scaffold protein. In the nanolipoprotein particle, the hydrophobically anchorable target molecule comprises an anchor moiety of approximately 2000 Da having a LogK_{ow} lower than 3.5 at conditions compatible with the integrity of the NLP structure, which associate to the lipid bilayer of the nanolipoprotein particle.

According to a fourth aspect, a method and system to provide a nanolipoprotein particle, are described. The method comprises contacting a membrane forming lipid and one or more lysolipids with a scaffold protein to provide a discoidal lipid bilayer comprising the membrane forming lipid and the one or more lysolipids stabilized by the scaffold protein.

The system comprises one or more membrane-forming lipids, one or more lysolipids, and a scaffold protein. In the system, assembly of the one or more membrane forming lipids and the scaffold protein provides a nanolipoprotein particle in which the one or more lysolipids are comprised within a membrane lipid bilayer stabilized by the scaffold protein.

According to a fifth aspect, a method and system to introduce a hydrophobically anchorable target molecule in a nanolipoprotein particle is described. The method comprises contacting the hydrophobically anchorable target molecule with a nanolipoprotein particle comprising a membrane lipid bilayer stabilized by a scaffold protein in a discoidal configuration with the membrane lipid bilayer comprising one or more lysolipids, to provide a nanolipoprotein particle comprising the hydrophobically anchorable target molecule within a membrane lipid bilayer.

The system comprises one or more hydrophobically anchorable target molecules, one or more nanolipoprotein particles comprising one or more lysolipids and one or more membrane forming lipids within a membrane lipid bilayer stabilized by a scaffold protein. In the system, contacting the one or more hydrophobically anchorable target molecules with the one or more nanolipoprotein particles provides the loaded nanolipoprotein particle herein described.

According to a sixth aspect, any one of the nanolipoprotein particle herein described comprises an active hydrophobically anchorable target molecule and/or further comprises an active target molecule, such as a drug or a molecule of interest, presented on the nanolipoprotein particle.

According to additional aspects, compositions, in particular pharmaceutical compositions and more particularly vaccines, methods and systems, comprising, forming and using the nanolipoprotein particles herein described are also provided in the present disclosure. Methods and systems to perform an assay on a hydrophobically anchorable target molecule and/or to deliver a hydrophobically anchorable target molecule based on the lysolipids-incorporating nanolipoprotein particles of the present disclosure are also described.

Nanolipoprotein particles herein described in several embodiments can increase the loading capacity of hydrophobically anchorable target molecules by a factor of 20-30 compared to the NLPs assembled in the absence of the lysolipids while maintaining the desired stability of the NLPs.

Nanolipoprotein particles herein described show in several embodiments a fraction by mass of the particle that is provided by a hydrophobically anchorable target molecule/cargo can increase from ≤1% to ≥10% compared to the NLPs not comprising lysolipids in the lipid bilayer.

Nanolipoprotein particles herein described show in several embodiments a loading capacity up to 30% of the total mass of the particle

Nanolipoprotein particles herein described show in several embodiments an ability to load target molecules that are hydrophobic and have a molecular weight up to 900 daltons or higher as incorporation of molecules in the kilodalton range is expected.

Nanolipoprotein particles and related compositions, methods and systems herein described can be used in connection with various applications wherein increased loading capacity of the NLP is desired. For example, the nanolipoprotein particles herein described and related compositions methods and systems can be used as a vehicle for delivery of compounds such as therapeutics to a specific target destination, as a platform for immunostimulating agents, vaccine development and use, and/or to contain cell-targeting moieties. Additional exemplary applications include uses of nanolipoprotein particles in several fields including basic biology research, applied biology, bio-engineering, bio-energy, molecular biology, medical research, medical diagnostics, therapeutics, bio-fuels, and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1 shows plots related to exemplary representative NLPs formed with lysophospholipids subsequently loaded with paclitaxel. FIG. 1, panel A is a plot showing representative SEC traces of NLPs prepared with lysophospholipid at 0 mol %, 40 mol % and 60 mol %. Two populations of NLPs are formed when lysophospholipids are including in the lipid mixture, which is observed in the 40 mol % sample. FIG. 1, panel B is a plot showing representative RP-HPLC traces of purified fractions from SEC of the 40 mol % NLPs depicted in FIG. 1, panel A. Lysophospholipids elute at 7.6 min. Differences in lysophospholipid content between the two purified peaks are observed. FIG. 1, panel C plots representative RP-HPLC traces showing the differences in paclitaxel loaded into the two NLP populations.

FIG. 2, panel A is a plot showing an exemplary amount of paclitaxel loaded into NLPs (mass of drug over total mass of particle components at the y-axis) as a function of initial lysophospholipid content (mol % at the x-axis). FIG. 2, panel B is a plot showing the amounts of each NLP component (at y-axis) measured for NLPs prepared with different initial lysophospholipid contents at 0 mol %, 30 mol % and 70 mol %.

FIG. 3 is a plot showing the amount of paclitaxel loaded into NLPs (at y-axis) as a function of lysophospholipid acyl chain structure (at x-axis). The y-axis is calculated as the mass of drug over total mass of particle components. The lysophospholipids of the x-axis are prepared at 50 mol % content.

### DETAILED DESCRIPTION

Provided herein are nanolipoprotein particles and related compositions, methods and systems.

The term “nanolipoprotein particle” “nanodisc” “rHDL” or “NLP” as used herein indicates a supramolecular complex formed by a membrane forming lipid arranged in a lipid bilayer stabilized by a scaffold protein. The membrane forming lipids and scaffold protein are components of the NLP. In particular the membrane forming lipid component is part of a total lipid component, (herein also membrane lipid component or lipid component) of the NLP together with additional lipids such as functionalized lipids and/or lysolipids, that can further be included in the NLPs as will be understood by a skilled person upon reading of the present disclosure. The scaffold protein component is part of a protein component of the NLP together with additional proteins such as membrane proteins, target proteins and other proteins that can be further included as components of the NLPs as will be understood by a skilled person upon reading of the present disclosure. Additional components can be provided as part of the NLP herein described as will be understood by a skilled person. In particular the membrane lipid bilayer can attach membrane proteins or other amphipathic compounds through interaction of respective hydrophobic regions with the membrane lipid bilayer. The membrane lipid bilayer can also attach proteins or other molecule through anchor compounds or functionalized lipids as will be understood by a skilled person upon reading of the disclosure. Predominately discoidal in shape, nanolipoprotein particles typically have diameters between 5 to 25 nm, share uniform heights between 3 to 6 nm and can be produced in yields ranging between 30 to 90%.

In particular in embodiments herein described the nanolipoprotein particle can be formed by a lipid bilayer confined in a discoidal configuration by a scaffold protein. In this configuration, the lipid bilayer confined by the scaffold protein can be 3-6 nanometers in thickness, the nanolipoprotein particle can have an overall diameter of 5-25 nanometers, and the scaffold protein on the particle can have a thickness of 1-2 nanometers. In some embodiments, an entire NLP structure can be up to 600 kilodaltons in weight.

The particular membrane forming lipid, scaffold protein, the lipid to protein ratio, and the assembly parameters determine the size and homogeneity of nanolipoprotein particles as will be understood by a skilled person. In the nanolipoprotein particle the membrane forming lipid are typically arranged in a membrane lipid bilayer confined by the scaffold protein in a discoidal configuration as will be understood by a skilled person.

The term “membrane forming lipid”, “amphipathic lipid”, or “polar lipid” as used herein indicates a lipid possessing both hydrophilic and hydrophobic moieties that in an aqueous environment assembles into a lipid bilayer structure that consists of two opposing layers of amphipathic molecules. Each polar or amphipathic lipid has a hydrophilic moiety, i.e. a polar group such as, a derivatized phosphate or a saccharide group, and a hydrophobic moiety, i.e., a long hydrocarbon chain. Exemplary types of polar or amphipathic lipids include phospholipids, sphingolipids, glycolipids, ether lipids, sterols, alkylphosphocholines and the like. Specific examples of polar lipids include but are not limited to phospholipids such as dimyristoylphosphatidylcholine (DMPC) or dioleoylphosphoethanolamine (DOPE) or dioleoylphosphatidylcholine (DOPC), or dipalmitoylphosphatidylcholine (DPPC). In a preferred embodiment, the lipid is dimyristoylphosphatidylcholine (DMPC).

The term “scaffold protein” as used herein indicates any amphipathic protein that is capable of self-assembly with amphipathic lipids in an aqueous environment, organizing the amphipathic lipids into a bilayer disc, and comprise apolipoproteins, lipophorins, derivatives thereof (such as truncated and tandemly arrayed sequences) and fragments thereof (e.g. peptides) which maintains the amphipathic nature and capability of self-assembly, such as apolipoprotein E4 (22Kd fragment), lipophorin III, apolipoprotein A-1 and the like. In general, scaffold proteins have an alpha helical secondary structure in which a plurality of hydrophobic amino acids form a hydrophobic face and a plurality of hydrophilic amino acids form an opposing hydrophilic face. In some embodiments, rationally designed amphipathic peptides and synthetic apolipoproteins which maintain an amphipathic structure and capability of self-assembly can serve as a scaffold protein of the NLP.

The term “apolipoprotein” as used herein indicates an amphipathic protein that binds lipids to form lipoproteins. The term “amphipathic” pertains to a molecule containing both hydrophilic and hydrophobic properties. Exemplary amphipathic molecules comprise molecules having hydrophobic and hydrophilic regions/portions in its structure. Examples of biomolecules which are amphipathic include but not limited to phospholipids, cholesterol, glycolipids, fatty acids, bile acids, saponins, and additional lipids identifiable by a skilled person. A “lipoprotein” as used herein indicates a biomolecule assembly that contains both proteins and lipids. In particular, in lipoproteins, the protein component surrounds or solubilizes the lipid molecules enabling particle formation. Exemplary lipoproteins include the plasma lipoprotein particles classified under high-density (HDL) and low-density (LDL) lipoproteins, which enable fats and cholesterol to be carried in the blood stream, the transmembrane proteins of the mitochondrion and the chloroplast, and bacterial lipoproteins. In particular, the lipid components of lipoproteins are insoluble in water, but because of their amphipathic properties, apolipoproteins such as certain Apolipoproteins A and Apolipoproteins B and other amphipathic protein molecules can organize the lipids in a bilayer orientation with exposed hydrophilic moieties, creating the lipoprotein particle that is itself water-soluble, and can thus be carried through water-based circulation (e.g. blood, lymph in vivo or in vitro). Apolipoproteins known to provide the protein components of the lipoproteins can be divided into six classes and several sub-classes, based on the different structures and functions. Exemplary apolipoprotein known to be able to form lipoproteins comprise Apolipoproteins A (apo A-I, apo A-II, apo A-IV, and apo A-V), Apolipoproteins B (apo B48 and apo B100), Apolipoproteins C (apo C-I, apo C-II, apo C-HI, and apo C-IV), Apolipoproteins D, Apolipoproteins E, and Apolipoproteins H. For example apolipoproteins B can form low-density lipoprotein particles, and have mostly beta-sheet structure and associate with lipid droplets irreversibly, while Apolipoprotein A1 comprise alpha helices and can associate with lipid droplets reversibly forming high-density lipoprotein particles.

The term “protein” as used herein indicates a polypeptide with a particular secondary and tertiary structure that can interact with another molecule and in particular, with other biomolecules including other proteins, DNA, RNA, lipids, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full-length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. In particular, in a protein, the polypeptide provides the primary structure of the protein, wherein the term “primary structure” of a protein refers to the sequence of amino acids in the polypeptide chain covalently linked to form the polypeptide polymer. A protein “sequence” indicates the order of the amino acids that form the primary structure. Covalent bonds between amino acids within the primary structure can include peptide bonds or disulfide bonds, and additional bonds identifiable by a skilled person. Polypeptides in the sense of the present disclosure are usually composed of a linear chain of alpha-amino acid residues covalently linked by peptide bond or a synthetic covalent linkage. The two ends of the linear polypeptide chain encompassing the terminal residues and the adjacent segment are referred to as the carboxyl terminus (C-terminus) and the amino terminus (N-terminus) based on the nature of the free group on each extremity. Unless otherwise indicated, counting of residues in a polypeptide is performed from the N-terminal end (NH₂-group), which is the end where the amino group is not involved in a peptide bond to the C-terminal end (—COOH group) which is the end where a COOH group is not involved in a peptide bond. Proteins and polypeptides can be identified by x-ray crystallography, direct sequencing, immunoprecipitation, and a variety of other methods as understood by a person skilled in the art. Proteins can be provided in vitro or in vivo by several methods identifiable by a skilled person. In some instances where the proteins are synthetic proteins in at least a portion of the polymer two or more amino acid monomers and/or analogs thereof are joined through chemically-mediated condensation of an organic acid (—COOH) and an amine (—NH₂) to form an amide bond or a “peptide” bond.

As used herein the term “amino acid”, “amino acid monomer”, or “amino acid residue” refers to organic compounds composed of amine and carboxylic acid functional groups, along with a side-chain specific to each amino acid. In particular, alpha- or α-amino acid refers to organic compounds composed of amine (—NH₂) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to form a polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D and L optical isomers.

In embodiments herein described, nanolipoprotein particles comprise one or more lysolipids within the lipid bilayer also comprising one or more membrane forming lipids.

The term “lysolipids” as used herein refers to a subset of polar lipids that are characterized by a single hydrocarbon chain and a polar head group and optionally a C1 to C6 additional aliphatic chain. Each lysolipid has a hydrophilic moiety (the polar head group which can be a derivatized phosphate or a saccharide group), and a hydrophobic moiety, (the single hydrocarbon chain).

Lysolipids in which the polar head group is a phosphate head group are identified as lysophospholipids. Two subgroups of lysophospholipids identifiable by a skilled person are: lysosphingolipids and lysoglycerophospholipids. Lysosphingolipids are lysophospholipids containing a sphingoid base backbone in the polar head group and lysoglycerophospholipids are lysophospholipids containing a glycerol backbone in the polar head group. In addition to the sphingoid base backbone or the glycerol backbone group, the polar head group of the lysophospholipids further comprises a phosphate group and another polar or charged group such as a choline, an ethanolamine, or a serine. The hydrocarbon chain presents variable lengths, degrees of saturation, degree of branching and other parameters that can alter the physical and biochemical properties of the lysophospholipid molecules.

In embodiments herein described lysolipids are included in the NLPs in ratios and amounts that is based on their surfactant effect, i.e. ability to solubilize lipids in an aqueous emulsion. Such feature allows lipids to behave as modulators of membrane fluidity and permeability. In particular, in embodiments herein described lysolipids are included with a critical micelle concentration ranging from 0.05 to 100 milliMolar.

The term “critical micelle concentration (CMC)” is used herein to characterize the aqueous solubility of a lipid compound. CMC indicates the concentration above which amphiphilic molecules aggregate to form micelles. At low surfactant concentration the amphiphilic molecules arrange on the surface of the liquid, but also exist as free molecules in the solution, with the two groups exchanging with one another. As more amphiphilic molecules are added, the surface becomes saturated, and the concentration of free molecules in solutions approaches a concentration specific to the molecule and environment, at which point further addition of amphiphilic molecules will lead to formation of micelles. This concentration point above which micelles form is called the critical micelle concentration. Thus, molecules that are more able to dissolve in solution without forming micelles have higher CMCs, while more insoluble molecules will have lower CMCs and will tend to form micelles with fewer molecules in solution.

Many factors have effects on the CMC of a lipid compound, for example, the molecular structure of the lipid, temperature, the presence of electrolyte in the solution and so on. The factors that contribute to CMC decrease of a lipid include but not limited to an increase in the number of carbon atoms in the hydrophobic tails, the existence of polyoxypropylene group, fluorocarbon structure, an increased degree of binding of the counterions, the existence of polar organic compounds (such as alcohols and amides) and others that can be identified by a skilled person in the art. The factors that contribute to CMC increase of a lipid include but not limited to branch hydrophobic structure, double bonds between carbon atoms, polar groups (O or OH) in hydrophobic tail, strongly ionized polar groups (sulphates and quaternaries), hydrophilic groups placed in the lipid molecule center, increase in the number of hydrophilic head, and other factors that can be identified by a skilled person in the art.

Several empirical correlations can be used for the estimation of CMC values. For lipids with a straight and saturated single carbon tail, the CMC can be calculated from (Klevens 1953):

Log CMC=A−Bn

where n is the number of carbon atoms in the hydrophobic tail, and A and B are temperature dependent constants for a given type of lipid. The values of A and B are identifiable for a skilled person in the art. These constant values can also be found in textbooks such as Kreshech 1975 [1]. It is also possible to measure the CMC of a molecule with such devices as a contact angle system, a tensiometer, a Langmuir trough, or with other equipment identifiable by a skilled person.

In some embodiments, lysolipids herein described comprise lysophospholipids of Formula (I):

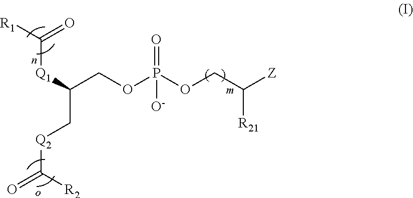

wherein R1 and R2 are independently selected from H, C1-C6 branched or linear substituted or unsubstituted aliphatic chain and a C7-C29 branched or linear, substituted or unsubstituted aliphatic chain;

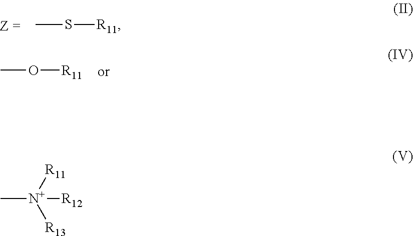

in which R₁₁, R₁₂ and R₁₃ are independently H or a C1-C4 branched or straight aliphatic chain;
R₂₁ is H, OH, or a carboxy group;
Q₁ and Q₂ are independently O, S, CH₂, NH, or NR₁₁;
m=0-3;
one of n and o is 0, and the other one of n and o is 1, and wherein one of R1 and R2 is the C7-C29 branched or straight, substituted or unsubstituted aliphatic chain and the other one of R1 and R2 is either H or the C1-C6 branched or linear substituted or unsubstituted aliphatic chain.

In some embodiments, the C1-C6 branched or linear substituted or unsubstituted aliphatic chain of formula (I) is a C1-C6 branched or linear substituted or unsubstituted heteroalkyl chain.

In some embodiments, lysolipids herein described comprise lysophospholipids of Formula (Ia):

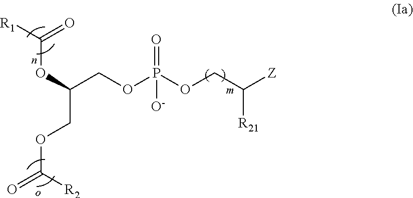

wherein R1 and R2 are independently selected from H, C1-C6 branched or linear, substituted or unsubstituted aliphatic chain and a C7-C29 branched or linear, substituted or unsubstituted aliphatic chain;

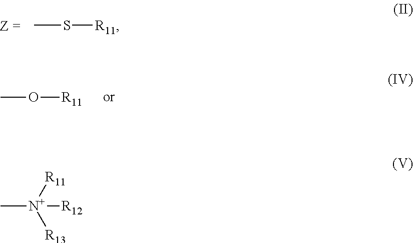

in which R₁₁, R₁₂ and R₁₁ are independently H or a C1-C4 branched or linear aliphatic chain;
R₂₁ is H, OH, or a carboxy group
m=0-3; and
one of n and o is 0, and the other one of n and o is 1, and
wherein one of R1 and R2 is the C7-C29 branched or linear, substituted or unsubstituted aliphatic chain and the other one of R1 and R2 is either H or the C1-C6 branched or linear, substituted or unsubstituted aliphatic chain.

In particular in some embodiments, one of R1 and R2 is H; n and o are independently 0 or 1; and when R1 is H, n is 0, when R2 is H, o is 0.

As used herein, the term “aliphatic” refers to that is an alkyl, alkenyl or alkynyl group which can be substituted or unsubstituted, linear, branched or cyclic.

As used herein the term “alkyl” as used herein refers to a linear, branched, or cyclic, saturated hydrocarbon group formed by a carbon chain. As used herein the term “carbon chain” indicates a linear or branched line of connected carbon atoms. An alkyl carbon chain typically although not necessarily containing 1 to about 18 carbon atoms, preferably 1 to about 6 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 30 carbon atoms. The term “cycloalkyl” intends a cyclic alkyl group, typically having 4 to 8, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. For example, a C12 heteroalkyl group refers to an alkyl group in which at least one of the carbon atom is replaced with a heteroatom and the total number of carbon and the heteroatom is 12. Thus the C12 heteroalkyl group has the same number of atoms as a C12 alkyl group, not including hydrogen. If not otherwise indicated, the term “alkyl” includes linear, branched, cyclic, unsubstituted, substituted, and/or heteroatom-containing alkyl groups. If not otherwise indicated, the term “lower alkyl” includes C1-C6 linear, branched, cyclic, unsubstituted, substituted, and/or heteroatom-containing alkyl groups.

As used herein the term “alkenyl” indicates a linear, branched, or cyclic hydrocarbon group that contains at least one carbon-carbon double bond. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. For example, a C12 heteroalkenyl group refers to an alkenyl group in which at least one of the carbon atom is replaced with a heteroatom and the total number of carbon and the heteroatom is 12. Thus the C12 heteroalkenyl group has the same number of atoms as a C12 alkenyl group, not including hydrogen. If not otherwise indicated, the term “alkenyl” includes linear, branched, cyclic, unsubstituted, substituted, and/or heteroatom-containing alkenyl groups.

If not otherwise indicated, the term “lower alkenyl” includes C1-C6 linear, branched, cyclic, unsubstituted, substituted, and/or heteroatom-containing alkenyl groups.

As used herein the term “alkynyl” indicates a linear, branched, or cyclic hydrocarbon group that contains at least one carbon-carbon triple bond. The term “substituted alkynyl” refers to alkenyl substituted with one or more substituent groups, and the terms “heteroatom-containing alkynyl” and “heteroalkenyl” refer to alkynyl in which at least one carbon atom is replaced with a heteroatom. For example, a C12 heteroalkynyl group refers to an alkynyl group in which at least one of the carbon atom is replaced with a heteroatom and the total number of carbon and the heteroatom is 12. Thus the C12 heteroalkynyl group has the same number of atoms as a C12 alkynyl group, not including hydrogen. If not otherwise indicated, the term “alkynyl” includes linear, branched, cyclic, unsubstituted, substituted, and/or heteroatom-containing alkynyl groups.

If not otherwise indicated, the term “lower alkynyl” includes C1-C6 linear, branched, cyclic, unsubstituted, substituted, and/or heteroatom-containing alkynyl groups.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 12 carbon atoms, and particularly preferred aryl groups contain 5 to 6 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, such as nitrogen, oxygen or sulfur.

As used herein the terms “heteroatom-containing” or “hetero-” indicated in connection with a group, refers to a hydrocarbon group in which one or more carbon atoms is replaced with an atom other than carbon, e.g., nitrogen, oxygen, sulfur, phosphorus or silicon, typically nitrogen, oxygen or sulfur. Exemplary “heteroatoms” comprise as N, O, S and P, and can be present in a compound by a covalent bond to each of two carbon atoms, thus interrupting the two carbon atoms. Accordingly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, and addition group identifiable by a skilled person.

The term “aralkyl” as used herein refers to an alkyl group with an aryl substituent, and the term “alkaryl” as used herein refers to an aryl group with an alkyl substituent, wherein “aryl” and “alkyl” are as defined above. In some embodiments, alkaryl and aralkyl groups contain 6 to 12 carbon atoms, and particularly alkaryl and aralkyl groups contain 6 to 16 carbon atoms. Alkaryl groups include, for example, p-methylphenyl, 2,4-dimethylphenyl, p-cyclohexylphenyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, and the like. The terms “alkaryloxy” and “aralkyloxy” refer to substituents of the formula —OR wherein R is alkaryl or aralkyl, respectively, as defined.

The terms “cyclic” and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic.

Unless otherwise indicated, the term “substituted” as in “substituted alkyl,” “substituted aryl,” and the like, is meant that in the, alkyl, aryl, or other moiety, at least one hydrogen atom bound to a carbon (or other) atom is replaced with one or more non-hydrogen substituents. As used herein, a “substituent” is an atom or group of atoms substituted in place of a hydrogen atom on the main chain of a hydrocarbon. Examples of such substituents include, without limitation: functional groups such as, hydroxyl, sulfhydryl, C₁-C₁₂ alkoxy, C₂-C₁₂ alkenyloxy, C₂-C₁₂ alkynyloxy, C₅-C₁₂ aryloxy, C₆-C₁₂ aralkyloxy, C₆-C₁₂ alkaryloxy, acyl (including C₂-C₁₂ alkylcarbonyl (—CO-alkyl) and C₆-C₁₂ arylcarbonyl (—CO-aryl)), acyloxy (—O-acyl, including C₂-C₁₂ alkylcarbonyloxy (—O—CO-alkyl) and C₆-C₁₂ arylcarbonyloxy (—O—CO-aryl)), C₂-C₁₂ alkoxycarbonyl (—(CO)—O— alkyl), C₆-C₁₂ aryloxycarbonyl (—(CO)—O-aryl), C₂-C₁₂ alkylcarbonato (—O—(CO)—O-alkyl), C₆-C₁₂ arylcarbonato (—O—(CO)—O-aryl), carboxy (—COOH), carboxylato (—COO—), carbamoyl (—(CO)—NH₂), mono-(C₁-C₁₂ alkyl)-substituted carbamoyl (—(CO)—NH(C₁-C₁₂ alkyl)), di-(C₁-C₁₂ alkyl)-substituted carbamoyl (—(CO)—N(C₁-C₁₂ alkyl)₂), mono-(C₅-C₁₂ aryl)-substituted carbamoyl (—(CO)—NH-aryl), di-(C₅-C₁₂ aryl)-substituted carbamoyl (—(CO)—N(C₅-C₁₂ aryl)₂), di-N—(C₁-C₆ alkyl), N—(C₅-C₁₂ aryl)-substituted carbamoyl, thiocarbamoyl (—(CS)—NH₂), mono-(C₁-C₁₂ alkyl)-substituted thiocarbamoyl (—(CO)—NH(C₁-C₁₂ alkyl)), di-(C₁-C₁₂ alkyl)-substituted thiocarbamoyl (—(CO)—N(C₁-C₆ alkyl)₂), mono-(C₅-C₁₂ aryl)-substituted thiocarbamoyl (—(CO)—NH-aryl), di-(C₅-C₆ aryl)-substituted thiocarbamoyl (—(CO)—N(C₅-C₆ aryl)₂), di-N—(C₁-C₆ alkyl), N—(C₅-C₆ aryl)-substituted thiocarbamoyl, carbamido (—NH—(CO)—NH₂), cyano(—C≡N), cyanato (—O—C≡N), thiocyanato (—S—C≡N), formyl (—(CO)—H), thioformyl (—(CS)—H), amino (—NH₂), mono-(C₁-C₁₂ alkyl)-substituted amino, di-(C₁-C₁₂ alkyl)-substituted amino, mono-(C₅-C₁₂ aryl)-substituted amino, di-(C₅-C₆ aryl)-substituted amino, C₂-C₁₂ alkylamido (—NH—(CO)-alkyl), C₆-C₁₂ arylamido (—NH—(CO)-aryl), imino (—CR═NH where R=hydrogen, C₁-C₁₂ alkyl, C₅-C₁₂ aryl, C₆-C₁₂ alkaryl, C₆-C₁₂ aralkyl, etc.), C₂-C₁₂ alkylimino (—CR═N(alkyl), where R=hydrogen, C₁-C₁₂ alkyl, C₅-C₁₂ aryl, C₆-C₁₂ alkaryl, C₆-C₂ aralkyl, etc.), arylimino (—CR═N(aryl), where R=hydrogen, C₁-C₁₂ alkyl, C₅-C₁₂ aryl, C₆-C₁₂ alkaryl, C₆-C₁₂ aralkyl, etc.), nitro (—NO₂), nitroso (—NO), sulfo (—SO₂—OH), sulfonato (—SO₂—O—), C₁-C₁₂ alkylsulfanyl (—S-alkyl; also termed “alkylthio”), C₅-C₁₂ arylsulfanyl (—S-aryl; also termed “arylthio”), C₁-C₁₂ alkylsulfinyl (—(SO)-alkyl), C₅-C₁₂ arylsulfinyl (—(SO)-aryl), C₁-C₁₂ alkylsulfonyl (—SO₂-alkyl), C₅-C₁₂ arylsulfonyl (—SO₂-aryl), boryl (—BH₂), borono (—B(OH)₂), boronato (—B(OR)₂ where R is alkyl or other hydrocarbyl), phosphono (—P(O)(OH)₂), phosphonato (—P(O)(O⁻)₂), phosphinato (—P(O)(O)), phospho (—PO₂), phosphino (—PH₂), silyl (—SiR₃ wherein R is hydrogen or hydrocarbyl), and silyloxy (—O-silyl); and the hydrocarbyl moieties C₁-C₁₂ alkyl (preferably C₁-C₁₂ alkyl, more preferably C₁-C₆ alkyl), C₂-C₁₂ alkenyl (preferably C₂-C₁₂ alkenyl, more preferably C₂-C₆ alkenyl), C₂-C₁₂ alkynyl (preferably C₂-C₁₂ alkynyl, more preferably C₂-C₆ alkynyl), C₅-C₁₂ aryl (preferably C₅-C₁₂ aryl), C₆-C₁₂ alkaryl (preferably C₆-C₁₂ alkaryl), and C₆-C₁₂ aralkyl (preferably C₆-C₁₂ aralkyl).

In some embodiments, the lysolipids comprise lipids of Formula (VI)

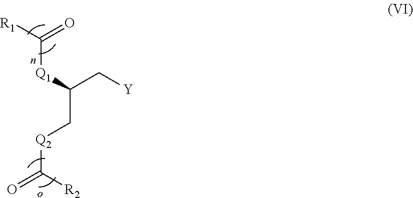

wherein R₁, R₂ are independently a H, C1-C6 branched or linear, substituted or unsubstituted aliphatic chain or C7-C29 branched or straight, substituted or unsubstituted aliphatic chain;

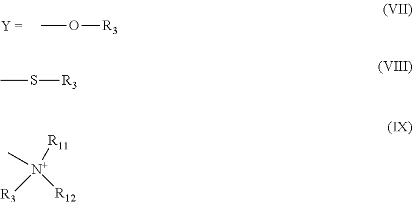

in which R₁₁, R₁₂, R₃ are independently H or a C1-C4 branched or straight aliphatic chain, one of n and o is 0, and the other one of n and o is 1;
Q₁ and Q₂ are independently O, S, CH₂, NH, or NR₁₁;
wherein one of R1 and R2 is the C7-C29 branched or straight, substituted or unsubstituted aliphatic chain and the other one of R1 and R2 is either H or the C1-C6 branched or linear, substituted or unsubstituted aliphatic chain.

In some embodiments, one of R1 and R2 is H; n and o is 0 or 1; and when R1 is H, n is 0, when R2 is H, o is 0.

In some embodiments, the lysolipids comprise lipids of Formula (Via)

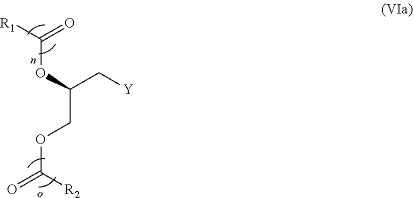

wherein R₁, R₂ are independently a H, C1-C6 branched or linear, substituted or unsubstituted aliphatic chain or C7-C29 branched or straight, substituted or unsubstituted aliphatic chain;

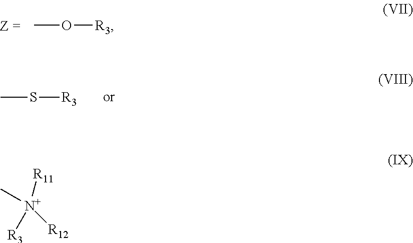

in which R₁₁, R₁₂, R₃ are independently H or a C1-C4 branched or straight aliphatic chain, one of n and o is 0, and the other one of n and o is 1
wherein one of R1 and R2 is the C7-C29 branched or straight, substituted or unsubstituted aliphatic chain and the other one of R1 and R2 is either H or the C1-C6 branched or linear, substituted or unsubstituted aliphatic chain.

In some embodiments, one of R1 and R2 is H; n and o is 0 or 1: and when R1 is H, n is 0, when R2 is H, o is 0.

In some embodiments, lysolipids comprise lipids of Formula (X)

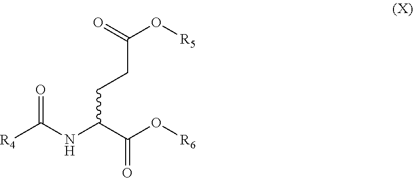

wherein R₄ is C7-C29 branched or straight, substituted or unsubstituted aliphatic chain,
R₅ and R₆ are independently H, C1-C6 branched or linear, substituted or unsubstituted aliphatic chain, or C1-C6 branched or linear aliphatic chain substituted with at least one amino nitrogen,
wherein one of R₅ and R₆ is a C1-C6 branched or linear aliphatic chain substituted with at least one amino nitrogen.

In some embodiments, lysolipids comprise lysosphingolipids having Formula (XI)

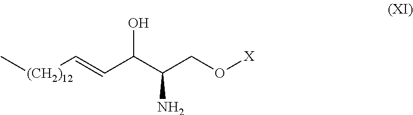

in which

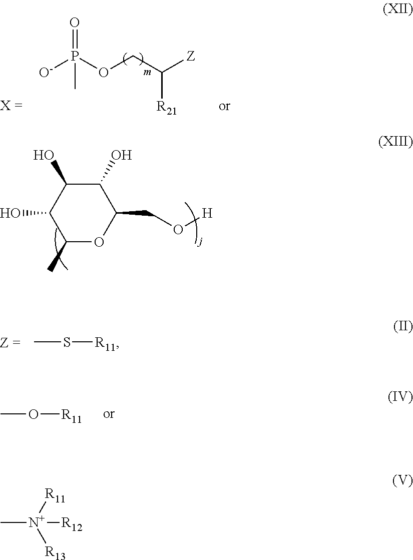

R₁₁, R₁₂ and R₁₃ are independently H or a C1-C4 branched or straight aliphatic chain;
R₂₁ is H, OH, or a carboxy group; and
m=0-3; and j=0-5.

In embodiments herein described, NLP comprise scaffold protein and a lipid component comprising lysolipids and membrane forming lipids in ratios and proportions that would be identifiable by a skilled person upon reading of the present disclosure.

In some embodiments, NLPs herein described have a lipid component to scaffold molar ratio ranging from 20:1 to 240:1, depending on the scaffold protein and the lipid component used as will be understood by a skilled person. For example, in NLPs herein described having apoE422k variants as scaffold protein and DOPC as the membrane forming lipid, the molar ratios of lipid component: scaffold protein component can range from 40:1 to 240:1 where the lipid molar ratios (membrane forming lipid to lysolipids) within the lipid component of the NLP can range from 9:1 to 3:7.

In some preferred embodiments, in the lysolipids-incorporating NLPs herein described, the molar ratios of lipid component: scaffold protein component is 80:1 (see Example 3).

In some embodiments, NLPs herein described have a lipid component comprising membrane forming lipids in an amount from 90 to 30 mol % of the lipid component and the lysolipids in an amount from 10 to 70 mol % of the lipid component. In some embodiments, the NLP herein described comprise membrane forming lipid and lysolipids in molar ratios ranging from 40 to 240.

In preferred embodiments, the NPLs herein described can have a membrane forming lipid:lysolipids:scaffold protein ratio range between 18:2:1 and 72:168:1, preferably 40:40:1, with exact molar ratios depending on the optimal lipid:protein ratio for that lipid mixture and scaffold protein identifiable by a skilled person upon reading of the present disclosure.

In some embodiments, the lysolipids are comprised within an NLP herein described in a molar lipid concentration about 10-70%, preferably about 50%. In some embodiments, the lysolipids can be comprised within an NLP herein described in an amount of 50% of the total lipid content of the NLP and the membrane forming lipids can be comprised within an NLP herein described in an amount of 50% of the total lipid content.

In some embodiments, the membrane forming lipids component of the lipid component lipids such as phospholipids, preferably including at least one phospholipid, typically soy phosphatidylcholine, egg phosphatidylcholine, soy phosphatidylglycerol, egg phosphatidylglycerol, palmitoyl-oleoyl-phosphatidylcholine distearoylphosphatidylcholine, or distearoylphosphatidylglycerol. Other useful phospholipids include, e.g., phosphatidylcholine, phosphatidylglycerol, sphingomyelin, phosphatidylserine, phosphatidic acid, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, stearoyl-palmitoyl-phosphatidylcholine, di-palmitoyl-phosphatidylethanolamine, distearoyl-phosphatidylethanolamine, di-myrstoyl-phosphatidylserine, and dioleyl-phosphatidylcholine.

Additionally exemplary membrane forming lipids that can be comprised in various combinations together with one or more lysolipids comprise 1,2-dipalmitoyl-sn-glycero-3-phosphocholine, 1,2-dimyristoyl-sn-glycero-3-phosphocholine, 1,2-dilauroyl-sn-glycero-3-phosphocholine, 1,2-didecanoyl-sn-glycero-3-phosphocholine, 1,2-dierucoyl-sn-glycero-3-phosphocholine, 1,2-dioleoyl-sn-glycero-3-phosphocholine, 1,2-dipalmitoleoyl-sn-glycero-3-phosphocholine, 1,2-dimyristoleoyl-sn-glycero-3-phosphocholine, 1-stearoyl-2-oleoyl-sn-glycero-3-phosphocholine, 1-stearoyl-2-myristoyl-sn-glycero-3-phosphocholine, 1-palmitoyl-2-oleoyl-sn-glycero-3-phosphocholine, egg phosphatidylcholine extracts, soy phosphatidylcholine extracts, heart phosphatidylcholine extracts, brain phosphatidylcholine extracts, liver phosphatidylcholine extracts, 1,2-distearoyl-sn-glycero-3-phosphate, 1,2-dipalmitoyl-sn-glycero-3-phosphate, 1,2-dimyristoyl-sn-glycero-3-phosphate, 1,2-dilauroyl-sn-glycero-3-phosphate, 1-palmitoyl-2-oleoyl-sn-glycero-3-phosphate, 1-stearoyl-2-oleoyl-sn-glycero-3-phosphate, 1,2-dipalmitoyl-sn-glycero-3-phosphoethanolamine, 1,2-dimyristoyl-sn-glycero-3-phosphoethanolamine, 1,2-dilauroyl-sn-glycero-3-phosphoethanolamine, 1,2-dioleoyl-sn-glycero-3-phosphoethanolamine, 1,2-dipalmitoleoyl-sn-glycero-3-phosphoethanolamine, Egg phosphatidylethanolamine extract, soy phosphatidylethanolamine extract, heart phosphatidylethanolamine extract, brain phosphatidylethanolamine extract, 1,2-distearoyl-sn-glycero-3-phospho-(1′-rac-glycerol), 1,2-dioleoyl-sn-glycero-3-phospho-(1′-rac-glycerol), 1,2-dipalmitoyl-sn-glycero-3-phospho-(1′-rac-glycerol), 1,2-dimyristoyl-sn-glycero-3-phospho-(1′-rac-glycerol), 1,2-dilauroyl-sn-glycero-3-phospho-(1′-rac-glycerol), 1-palmitoyl-2-oleoyl-sn-glycero-3-phospho-(1′-rac-glycerol), egg phosphatidylglycerol extract, soy phosphatidylglycerol extract, 1,2-distearoyl-sn-glycero-3-phospho-L-serine, 1,2-dioleoyl-sn-glycero-3-phospho-L-serine, 1,2-dipalmitoyl-sn-glycero-3-phospho-L-serine, 1,2-dimyristoyl-sn-glycero-3-phospho-L-serine, 1,2-dilauroyl-sn-glycero-3-phospho-L-serine, 1-palmitoyl-2-oleoyl-sn-glycero-3-phospho-L-serine, soy phosphatidylserine extract, brain phosphatidylserine extract, 2-((2,3-bis(oleoyloxy)propyl)dimethylammonio)ethyl hydrogen phosphate, cholesterol, ergosterol, sphingolipids, ceramides, sphingomyelin, gangliosides, glycosphingolipids, 1,2-dioleoyl-3-trimethyl ammonium-propane, 1,2-di-O-octadecenyl-3-trimethylammonium propane.

In some embodiments, non-phosphorus containing lipids can also be used as membrane forming lipids in the NLPs herein described, e.g. stearylamine, docecylamine, acetyl palmitate, and fatty acid amides. Additional membrane forming lipids suitable for use in providing NLPs are well known to persons of ordinary skill in the art and are cited in a variety of well-known sources, e.g., McCutcheon's Detergents and Emulsifiers and McCutcheon's Functional Materials, Allured Publishing Co., Ridgewood, N.J., both of which are incorporated herein by reference.

In some embodiments, the lysolipid component comprises lipids such as 1-hexanoyl-2-hydroxy-sn-glycero-3-phosphocholine (lyso 6:0), 1-heptanoyl-2-hydroxy-sn-glycero-3-phosphocholine (lyso 7:0), 1-octanoyl-2-hydroxy-sn-glycero-3-phosphocholine (lyso 8:0), 1-nonanoyl-2-hydroxy-sn-glycero-3-phosphocholine (lyso 9:0), 1-decanoyl-2-hydroxy-sn-glycero-3-phosphocholine (lyso 10:0), 1-undecanoyl-2-hydroxy-sn-glycero-3-phosphocholine (lyso 11:0), 1-lauroyl-2-hydroxy-sn-glycero-3-phosphocholine (lyso 12:0), 1-tridecanoyl-2-hydroxy-sn-glycero-3-phosphocholine (lyso 13:0), 1-tetradecanoyl-sn-glycero-3-phosphocholine (lyso 14:0), 1-pentadecanoyl-2-hydroxy-sn-glycero-3-phosphocholine (lyso 15:0), 1-hexadecanoyl-sn-glycero-3-phosphocholine (lyso 16:0), 1-heptadecanoyl-2-hydroxy-sn-glycero-3-phosphocholine (Lyso 17:0), 1-octadecanoyl-sn-glycero-3-phosphocholine lyso (18:0), 1-(9Z-octadecenoyl)-sn-glycero-3-phosphocholine (lyso 18:1).

In some embodiments various combinations and ratios of membrane forming lipids and lysolipids can be comprised within an NLP herein described, such as 1,2-dioleoyl-sn-glycero-3-phosphocholine (DOPC) and 1-hexanoyl-2-hydroxy-sn-glycero-3-phosphocholine (lyso 6:0) at ratio range of 9:1 to 3:7, 1,2-dioleoyl-sn-glycero-3-phosphocholine (DOPC) and 1-heptanoyl-2-hydroxy-s-glycero-3-phosphocholine (lyso 7:0) at ratio range of 9:1 to 3:7, 1-octanoyl-2-hydroxy-sn-glycero-3-phosphocholine (lyso 8:0) at ratio range of 9:1 to 3:7, 1,2-dioleoyl-sn-glycero-3-phosphocholine (DOPC) and 1-nonanoyl-2-hydroxy-sn-glycero-3-phosphocholine (lyso 9:0) at ratio range of 9:1 to 3:7, 1,2-dioleoyl-sn-glycero-3-phosphocholine (DOPC) and 1-decanoyl-2-hydroxy-sn-glycero-3-phosphocholine (lyso 10:0) at ratio range of 9:1 to 3:7, 1.2 dioleoyl-sn-glycero-3-phosphocholine (DOPC) and 1-undecanoyl-2-hydroxy-sn-glycero-3-phosphocholine (lyso 11:0) at ratio range of 9:1 to 3:7, 1,2-dioleoyl-sn-glycero-3-phosphocholine (DOPC) and 1-lauroyl-2-hydroxy-sn-glycero-3-phosphocholine (lyso 12:0) at ratio range of 9:1 to 3:7, 1,2-dioleoyl-sn-glycero-3-phosphocholine (DOPC) and 1-tridecanoyl-2-hydroxy-sn-glycero-3-phosphocholine (lyso 13:0) at ratio range of 9:1 to 3:7,1,2-dioleoyl-sn-glycero-3-phosphocholine (DOPC) and 1-tetradecanoyl-sn-glycero-3-phosphocholine (lyso 14:0) at ratio range of 9:1 to 3:7,1,2-dioleoyl-sn-glycero-3-phosphocholine (DOPC) and 1-pentadecanoyl-2-hydroxy-sn-glycero-3-phosphocholine (lyso 15:0) at ratio range of 9:1 to 3:7, 1,2-dioleoyl-sn-glycero-3-phosphocholine (DOPC) and 1-hexadecanoyl-sn-glycero-3-phosphocholine (lyso 16:0) at ratio range of 9:1 to 3:7, 1,2-dioleoyl-sn-glycero-3-phosphocholine (DOPC) and 1-heptadecanoyl-2-hydroxy-sn-glycero-3-phosphocholine (Lyso 17:0) at ratio range of 9:1 to 3:7, 1,2-dioleoyl-sn-glycero-3-phosphocholine (DOPC) and 1-octadecanoyl-sn-glycero-3-phosphocholine lyso (18:0) at ratio range of 9:1 to 3:7, 1,2-dioleoyl-sn-glycero-3-phosphocholine (DOPC) and 1-(9Z-octadecenoyl)-sn-glycero-3-phosphocholine (lyso 18:1) at ratio range of 9:1 to 3:7.

The above ratio between membrane forming lipid DOPC and various lysolipids can be derived for other membrane forming lipids such as DMPC, POPC, DOPE, DPPC and natural lipids extracts such as SoyPC and EggPC and a mixture thereof as will be understood for a skilled person, and can range from 9:1 to 3:7.

In some embodiments the scaffold proteins can contain amino acid additions, deletions, or substitutions. In other embodiments, the scaffold proteins can be derived from various species and more particularly derived from human, mouse, rat, guinea pig, rabbit, cow, horse, pig, dog, and non-human primates.

In some embodiments various combinations of membrane forming lipids and lysophospholipids in according with the disclosure can be comprised within an NLP stabilized by scaffold proteins such as human derived apoE4, truncated versions of human derived apoE4 (e.g. apoE422k), human derived apoE3, truncated versions of human derived apoE3 (e.g. apoE322k), human derived apoE2, truncated versions of human derived apoE2 (e.g. apoE222k), human derived apoA1, truncated versions of human derived apoA1 (e.g. Δ49ApoA1, MSP1, MSP1T2, MSP1E3D1), mouse derived apoE4, truncated versions of mouse derived apoE4 (e.g. apoE422k), mouse derived apoE3, truncated versions of mouse derived apoE3 (e.g. apoE322k), mouse derived apoE2, truncated versions of mouse derived apoE2 (e.g. apoE222k), mouse derived apoA1, truncated versions of mouse derived apoA1 (e.g. Δ49ApoA1, MSP1, MSP1T2, MSP1E3D1), rat derived apoE4, truncated versions of rat derived apoE4 (e.g. apoE422k), rat derived apoE3, truncated versions of rat derived apoE3 (e.g. apoE322k), rat derived apoE2, truncated versions of rat derived apoE2 (e.g. apoE222k), rat derived apoA1, truncated versions of rat derived apoA1 (e.g. Δ49ApoA1, MSP1, MSP1T2, MSP1E3D1), lipophorins (e.g. B. mori, M. sexta), synthetic cyclic peptides that mimic the function of apolipoproteins. Other apolipoproteins, as will be understood for a skilled person, can be used to form NLP, including but not limited to apoB and apoC.

In some embodiments various combinations of membrane forming lipids and lysolipids in accordance with the disclosure can be comprised within an NLP stabilized by different scaffold proteins, 1,2-dioleoyl-sn-glycero-3-phosphocholine (DOPC) and 1-hexanoyl-2-hydroxy-sn-glycero-3-phosphocholine (lyso 6:0) (ratio range of 9:1 to 3:7) with human derived apoE422k (lipid:scaffold protein range 40:1 to 200:1), 1,2-dioleoyl-sn-glycero-3-phosphocholine (DOPC) and 1-heptanoyl-2-hydroxy-sn-glycero-3-phosphocholine (lyso 7:0) (ratio range of 9:1 to 3:7) with human derived apoE422k (lipid:scaffold protein range 40:1 to 200:1), 1-octanoyl-2-hydroxy-sn-glycero-3-phosphocholine (lyso 8:0) (ratio range of 9:1 to 3:7) with human derived apoE422k (lipid:scaffold protein range 40:1 to 200:1), 1,2-dioleoyl-sn-glycero-3-phosphocholine (DOPC) and 1-nonanoyl-2-hydroxy-sn-glycero-3-phosphocholine (lyso 9:0) (ratio range of 9:1 to 3:7) with human derived apoE422k (lipid:scaffold protein range 40:1 to 200:1), 1,2-dioleoyl-sn-glycero-3-phosphocholine (DOPC) and 1-decanoyl-2-hydroxy-sn-glycero-3-phosphocholine (lyso 10:0) (ratio range of 9:1 to 3:7) with human derived apoE422k (lipid:scaffold protein range 40:1 to 200:1), 1,2-dioleoyl-sn-glycero-3-phosphocholine (DOPC) and 1-undecanoyl-2-hydroxy-sn-glycero-3-phosphocholine (lyso 11:0) (ratio range of 9:1 to 3:7) with human derived apoE422k (lipid:scaffold protein range 40:1 to 200:1), 1,2-dioleoyl-sn-glycero-3-phosphocholine (DOPC) and 1-lauroyl-2-hydroxy-sn-glycero-3-phosphocholine (lyso 12:0) (ratio range of 9:1 to 3:7) with human derived apoE422k (lipid:scaffold protein range 40:1 to 200:1), 1,2-dioleoyl-sn-glycero-3-phosphocholine (DOPC) and 1-tridecanoyl-2-hydroxy-sn-glycero-3-phosphocholine (lyso 13:0) (ratio range of 9:1 to 3:7) with human derived apoE422k (lipid:scaffold protein range 40:1 to 200:1), 1,2-dioleoyl-sn-glycero-3-phosphocholine (DOPC) and 1-tetradecanoyl-sn-glycero-3-phosphocholine (lyso 14:0) (ratio range of 9:1 to 3:7) with human derived apoE422k (lipid:scaffold protein range 40:1 to 200:1), 1,2-dioleoyl-sn-glycero-3-phosphocholine (DOPC) and 1-pentadecanoyl-2-hydroxy-sn-glycero-3-phosphocholine (lyso 15:0) (ratio range of 9:1 to 3:7) with human derived apoE422k (lipid:scaffold protein range 40:1 to 200:1), 1,2-dioleoyl-sn-glycero-3-phosphocholine (DOPC) and 1-hexadecanoyl-sn-glycero-3-phosphocholine (lyso 16:0) (ratio range of 9:1 to 3:7) with human derived apoE422k (lipid:scaffold protein range 40:1 to 200:1), 1,2-dioleoyl-sn-glycero-3-phosphocholine (DOPC) and 1-heptadecanoyl-2-hydroxy-sn-glycero-3-phosphocholine (Lyso 17:0) (ratio range of 9:1 to 3:7) with human derived apoE422k (lipid:scaffold protein range 40:1 to 200:1), 1,2-dioleoyl-sn-glycero-3-phosphocholine (DOPC) and 1-octadecanoyl-sn-glycero-3-phosphocholine lyso (18:0) (ratio range of 9:1 to 3:7) with human derived apoE422k (lipid:scaffold protein range 40:1 to 200:1), 1,2-dioleoyl-sn-glycero-3-phosphocholine (DOPC) and 1-(9Z-octadecenoyl)-sn-glycero-3-phosphocholine (lyso 18:1) (ratio range of 9:1 to 3:7) with human derived apoE422k (lipid:scaffold protein range 40:1 to 200:1).

The above ratios between membrane forming lipid DOPC, various lysolipids, and human derived apoE422k can be derived for other membrane forming lipids such as DMPC, POPC, DOPE, DPPC and natural lipids extracts such as SoyPC and EggPC and a mixture thereof as will be understood for a skilled person upon the reading of the present disclosure. [2]

The above ratios between lipids (membrane forming and various lysolipids) and apoE-derived scaffold proteins can be derived for other membrane forming lipids such as DMPC, POPC, DOPE, DPPC and natural lipids extracts such as SoyPC and EggPC and a mixture thereof as will be understood for a skilled person, and can range from 9:1 to 3:7.

In some embodiments, the ratios between lipids (membrane forming and various lysolipids) and apoA-derived scaffold proteins can be derived for other membrane forming lipids such as DOPC, DMPC, POPC, DOPE, DPPC and natural lipids extracts such as SoyPC and EggPC and a mixture thereof as will be understood for a skilled person, and can range from 20:1 to 180:1.

In embodiments, herein described lysolipids-incorporating NLPs can be prepared with various methods resulting in the assembly of the lipid component formed by the membrane forming lipid and the lysolipids with the scaffold protein.

In particular, in some embodiments the lysolipids-NLP lipid component and scaffold protein component can be contacted to form an admixture for a time and under conditions allowing assembling of the NLP according to methods known or identifiable by a skilled person upon reading of the present disclosure.

For example in some embodiments, NLPs herein described can be assembled by a dialysis method, which is a self-assembly process involving detergent solubilization of lipids followed by detergent removal as described for example in [3-5]. A dialysis method typically involves solubilizing the membrane lipid component in a detergent, such as sodium cholate, at detergent concentrations above the critical micelle concentration. The resulting lipid/detergent solution is then incubated to allow for dissolution of the scaffold protein and sufficient interaction between the scaffold protein and lipid mixture (e.g. for about 30 min). After the incubation period, the detergent is removed (e.g. through dialysis or rinsing with detergent binding beads) and the scaffold protein of choice is added at an appropriate lipid to apolipoprotein ratio that will allow for self-assembly as will be understood by a skilled person upon reading of the present disclosure. In particular, the NLP typically self-assemble during the detergent removal process.

For example, in some embodiments, NLPs herein described can be assembled following a detergent-binding bead method, which is a self-assembly process involving detergent solubilization of lipids followed by detergent removal. This method typically involves solubilizing the membrane lipid component in a detergent, such as sodium cholate, at detergent concentrations above the critical micelle concentration. The resulting lipid/detergent solution is then incubated to allow for dissolution of the scaffold protein and sufficient interaction between the scaffold protein and lipid mixture (e.g. for about 30 min). After the incubation period, the detergent is removed by incubating with detergent binding beads and the scaffold protein of choice is added at an appropriate lipid to apolipoprotein ratio that will allow for self-assembly as will be understood by a skilled person upon reading of the present disclosure. In particular, the NLPs typically self-assemble during the detergent removal process. An example of a detergent commonly used to prepare apolipoprotein-lipid complexes is sodium cholate.

In some embodiments, NLPs herein described can be assembled by temperature cycling method, where an admixture of lipid component and scaffold protein component forming the NLPs that is subjected to a temperature transition cycle in presence of a detergent such as the one described in [6-8]. In the temperature cycle, the temperature of the admixture is raised above and below the gel crystalline transition temperature of the membrane forming lipids. In particular, in accordance with an exemplary procedure the lipid component including membrane forming lipid and lysolipids can be added to the scaffold protein at the desired lipid to scaffold protein ratio in buffer. After thoroughly mixing the components, the solution is incubated a particular temperature for a certain amount of time. For example, the solution can be maintained at 23.8° C. for at least 2 hours (see Example 3).

In some embodiments, NLPs herein described can be assembled by an in vitro translation method, where self-assembly of the NLPs can be achieved while the apolipoprotein or other scaffold protein is being translated from mRNA as described for example in [9-12]. In this process, expression system lysates are mixed with the lipid component of the NLP and plasmid DNA encoding the scaffold protein. The reaction can then be allowed to proceed until assembly occurs during apolipoprotein expression (e.g. for approximately 4-24 hrs). The apolipoprotein typically contains an affinity tag (e.g. His-tag) for subsequent purification of the self-assembled NLP from the lysate.

In general, assembly of lysolipids-incorporating NLPs can be accomplished with a wide range of ratios of total membrane forming lipids to scaffold proteins. Lysolipids-incorporating NLPs with lipid to scaffold molar ratios of about 20:1 up to about 240:1 can be synthesized. A typical assembly with apoE and DOPC uses a lipid to protein molar ratio of about 80:1.

In some embodiments, the methods and systems herein described are performed at predefined lipid protein ratio, assembly conditions and/or with the use of preselected protein component and lipid component so to increase the yield, control the size and composition of the resulting NLP, provide an NLP of pre-determined dimensions, achieve desired functionality of the NLP, such as a certain level of loading capacity for a target drug molecule. In some embodiments, the molar ratio of lipid component to scaffold protein component is 20:1, 30:1, 40:1, 50:1, 60:1, 70:1, 80:1, 90:1, 100:1, 110:1, 120:1, 130:1, 140:1, 150:1, 160:1, 170:1, 180:1, 190:1, 200:1, 210:1, 220:1, 230:1, and 240:1. In NLPs herein described, the lipid to scaffold protein component ratio can be determined on a case by case basis in view of the experimental design as will be understood by a skilled person.

Composition of an NLP can be detected by various techniques known in the art, such as high performance liquid chromatography (HPLC), reverse phase high performance liquid chromatography (RP-HPLC), mass spectrometry, thin layer chromatography, NMR spectroscopy and elemental analysis could be used to define the composition of the particles and additional techniques identifiable by a skilled person.

In several embodiments herein described, lysolipids-incorporating NLPs show different size and compositions (see Examples 4-5). Size and compositions of the resulting NLPs can be characterized by SEC (size exclusion chromatography) traces which are used to separate out molecules in solution by their size and in some cases their molecular weights as will be understood by a skilled person.

In several embodiments herein described, lysolipids-incorporating NLPs show an increased loading capacity for target drugs, in particular, hydrophobic drugs, such as paclitaxel. The mass of the loaded drug over the total mass of NLPs as well as the mass of the individual component of the NLPs, including the membrane forming lipids, lysolipids, scaffold proteins and other amphipathic compounds, can be characterized by the reverse phase HPLC as will be understood by a skilled person (see Example 4.)

In some embodiments, NLPs herein described can further include additional lipids such as polymerizable lipids, functionalized amphipathic compounds and/or one or more target proteins that can be added during the assembly of the NLP herein described also comprising one or more lysophospholipids.

The term “polymerizable lipid” as used herein indicates a lipid molecule comprising at least one functional group presented for reaction with a corresponding functional group in presence of a crosslinking agent or initiator to provide a polymer formed by two or more same or different lipid molecules. Polymerizable lipids herein described therefore present corresponding functional groups in a configuration allowing reaction of the corresponding functional groups upon introduction of a cross-linking agent or initiator to provide polymerized lipid molecules.

The term “functional group” as used herein indicates specific groups of atoms within a molecular structure that are responsible for a characteristic chemical reaction of that structure. Exemplary functional groups include hydrocarbons, groups containing double or triple bonds, groups containing halogen, groups containing oxygen, groups containing nitrogen and groups containing phosphorus and sulfur all identifiable by a skilled person.

Functional groups presented in polymerizable lipids to provide polymerized lipids (herein also polymerizable functional groups) can contain at least one double and/or triple bond that can react in presence of the crosslinking agent or initiator to provide the polymerized lipid comprising at least two polymerizable lipid bound to one another. In particular, in embodiments here described one or more polymerizable functional groups comprising one double and/or triple bond is located in the hydrophobic region of the polymerizable lipid molecule. More particularly polymerizable functional groups within polymerizable lipids comprise various groups (e.g. hydrocarbon group, a group containing oxygen, a group containing nitrogen and a group containing phosphorus and/or sulfur) presenting at least one double and/or triple bond. In particular, functional groups in the sense of the present disclosure include diacetylene groups [13, 14], methacrylate groups [15, 16], acryloyl groups [17, 18], sorbyl ester groups [19], diene groups [20, 21], styrene groups [22], vinyl groups [22] and isocyano groups [22]. Additional functional groups can be identified by a skilled person upon reading of the present disclosure.

The term “corresponding” used in connection with elements such as functional groups identify two or more elements capable of reacting one with another under appropriate conditions. Typically, a reaction between corresponding moieties and in particular functional groups, results in binding of the two elements.

The term “bind”, “binding”, “conjugation” as used herein indicates an attractive interaction between two elements which results in a stable association of the element in which the elements are in close proximity to each other. If each element is comprised in a molecule the result of binding is typically formation of a molecular complex. Attractive interactions in the sense of the present disclosure includes both non-covalent binding and, covalent binding. Covalent binding indicates a form of chemical bonding that is characterized by the sharing of pairs of electrons between atoms, or between atoms and other covalent bonds. For example, attraction-to-repulsion stability that forms between atoms when they share electrons is known as covalent bonding. Covalent bonding includes many kinds of interactions, including σ-bonding, π-bonding, metal to non-metal bonding, agostic interactions, and three-center two-electron bonds. Non-covalent binding as used herein indicates a type of chemical bond, such as protein-protein interaction, that does not involve the sharing of pairs of electrons, but rather involves more dispersed variations of electromagnetic interactions. Non-covalent bonding includes ionic bonds, hydrophobic interactions, electrostatic interactions, hydrogen bonds, and dipole-dipole bonds. Electrostatic interactions include association between two oppositely charged entities. An example of an electrostatic interaction includes using a charged lipid as the functional membrane lipid and binding an oppositely charged target molecule through electrostatic interactions.

Exemplary corresponding functional groups capable of reacting in presence of an initiator to provide polymerized lipids comprise diacetylene groups (initiator—UV exposure) [13, 14], methacrylate groups (initiator−UV exposure, azobisisobutyronitrile (AIBN)+heat) [15, 16], acryloyl groups (initiator−(AIBN)+heat) [17, 18], sorbyl ester groups (initiator−UV exposure, azobisisobutyronitrile (AIBN)+heat) [19], diene groups (initiator−UV exposure, azobisisobutyronitrile (AIBN)+heat, azobis(2-amidinopropane) dihydrochloride (AAPD)+heat) [20, 21], styrene groups (initiator−UV exposure) [22], vinyl groups (initiator−UV exposure) [22] and isocyano groups (initiator−UV exposure) [22]. In some embodiments, in polymerizable lipids herein described at least one polymerizable functional group is selected from diacetylenyl, acryloyl, methacryloyl and dienyl groups.

Polymerizable lipids in the sense of the disclosure comprises lipids used to provide stable multilayers of long chained fatty acids that display unique physical properties such as photoconductivity, photochemistry and photophysics [23], lipids used to stabilize planar lipid structures (see e.g. [24]), lipids used to form lipid assemblies in a variety of configurations (see e.g. [13], [14-15], [16]) as well as commercially available lipids including components in sensors [14, 25] and in vesicle-based drug-delivery vehicles [26-28]. One specific type of polymerizable lipid that is often used is based on long-chain diacetylene monocarboxylic acids, which have been well-studied and have been shown to form intermolecular covalent bonds as a result of exposure to ultraviolet light at 254 nanometers [23, 29, 30].

In some embodiments, the polymerizable lipid component comprise lipids such as 1,2-bis(10,12-tricosadiynoyl)-sn-glycero-3-phosphocholine (polymerizable group in both acyl chains), 1,2-bis(10,12-tricosadiynoyl)-sn-glycero-3-phosphoethanolamine (polymerizable group in both acyl chains), 1-palmitoyl-2-(10,12-tricosadiynoyl)-sn-glycero-3-phosphocholine (polymerizable group in one acyl chain), 1-palmitoyl-2-(10,12-tricosadiynoyl)-sn-glycero-3-phosphoethanolamine (polymerizable group in one acyl chain), rac-1-stearoyl-2-(octadeca-2,4-trans,trans-dienoyl)glycero-3-phosphorylcholine, rac-1,2-bis(octadeca-2,4-trans,trans-dienoyl)glycero-3-phosphorylcholine, bis(docosa-10,12-diynyl)N-[6-(triethylammonio)hexanoyl]-L-glutamate bromide, N-[11-(trimethylammonino)undecanoyl]-L-glutamate bromide, N-[4-(trimethylammonio)-butoxybenzoyl] glutamate bromide, 2,3-Bis(hexadecanoyloxy) propy]-9-methacryloyl-3,6,9-trioxanonyldimethylanimonium Iodide, 12-MethacryIoyl-3,6,9,12-tetraoxadodecyl 3-(N,N-Dioctadecylcarbamoyl) propionate, 2,3-Bis(hexadecyloxy)propyl 12-Methacryloyl-3,6,9,12-tetraoxadodecyl Succinate, Sodium 2,3-Bis(hexadecyloxy)propyl-12-methacryloyl-3,6,9,12-tetraoxadodecylphosphate.

In some embodiment, NLPs herein described can include polymerizable lipids having two polymerizable functional groups per lipid and the polymerizable lipids constitute 20% of the membrane-forming lipid mixture.

In some embodiments, NLPs herein described can further include 1,2-bis(10,12-tricosadiynoyl)-sn-glycero-3-phosphocholine (DiynePC). In some embodiments, DiynePC will preferably constitute 20 mol % of the membrane-forming lipid mixture.

In some embodiment, the lipid bilayer of nanolipoproteins herein described can further comprise one or more functionalized amphipathic compounds, which provide an additional component of the NLP herein described.

The term “functionalized amphipathic compounds” in the sense of the disclosure indicates compounds having a hydrophobic portion and hydrophilic portions in a configuration where the hydrophobic portion anchor is able to anchor the compound to the lipid bilayer of the NLP and the hydrophilic portion (typically consisting or comprising a hydrophilic functional group) is presented on the NLP bilayer face following NLP assembly.

The term “present” as used herein with reference to a compound or functional group indicates attachment performed to maintain the chemical reactivity of the compound or functional group as attached. Accordingly, a functional group presented on an amphipathic compound, is able to perform under the appropriate conditions the one or more chemical reactions that chemically characterize the functional group.

The use of functionalized amphipathic compounds enables attachment of various peptides or other biologics to the surfaces of the lipid of the NLP that allows some desired target features to be obtained, such as stability, affinity for a target molecule, and the like. Non-limiting examples of functional groups presented on functionalized lipids include: chelated Ni atoms, azide, anhydride, alkynes, thiols, halogens, carboxy, amino, hydroxyl, and phosphate groups, and the like.

In some embodiments, the functional group on the functionalized amphipathic compound can be a reactive chemical groups (e.g. azide, chelated nickel, alkyne, and additional reactive chemical groups identifiable by a skilled person), a biologically active compound (e.g. DNA, peptide, carbohydrate, and additional biologically active group identifiable by a skilled person) or a small molecule (e.g. cellular targeting compound, adjuvant, drug, and additional small molecules identifiable by a skilled person). In some embodiments the functionalized amphipathic compound is a functionalized lipid compound. Functional groups that enhance the lipid solubility are referred to as hydrophobic or lipophilic functional groups. Functional groups that lack the ability to either ionize or form hydrogen bonds tend to impart a measure of lipid solubility to a drug molecule. The functional group can be attached to the lipid polar head through covalent or ionic bonds and “weak bonds” such as dipole-dipole interactions, the London dispersion force and hydrogen bonding, preferably covalent. Moreover, functionalization of the lipid can involve hydrophobic quantum dots embedded into the lipid bilayer. The following article is incorporated by reference in its entirety: R. A. Sperling, and W. J. Parak. “Surface modification, functionalization and bioconjugation of colloidal inorganic nanoparticles”. Phil. Trans. R. Soc. A 28 Mar. 2010 vol. 368 no. 1915 1333-1383.

In some embodiments, functionalized amphipathic compounds can comprise one or more of 1,2-dipalmitoyl-sn-glycero-3-phosphoethanolamine-N-(6-((folate)amino)hexanoyl), 1,2-dipalmitoyl-sn-glycero-3-phosphoethanolamine-N-(6-azidohexanoyl), 1,2-dipalmitoyl-sn-glycero-3-phosphoethanolamine-N-(succinyl), 1,2-dioleoyl-sn-glycero-3-phosphoethanolamine-N-(succinyl), 1,2-dipalmitoyl-sn-glycero-3-phosphoethanolamine-N-(glutaryl), 1,2-dioleoyl-sn-glycero-3-phosphoethanolamine-N-(glutaryl), 1,2-dioleoyl-sn-glycero-3-phosphoethanolamine-N-(dodecanyl), 1,2-Dipalmitoyl-sn-Glycero-3-Phosphoethanolamine-N-(hexanoylamine), 1,2-dipalmitoyl-sn-glycero-3-phosphoethanolamine-N-(dodecanylamine), 1,2-Dipalmitoyl-sn-Glycero-3-Phosphothioethanol, 1,2-di oleoyl-sn-glycero-3-phosphoethanol amine-N-[4-(p-maleimidomethyl)cyclohexane-carboxamide], 1,2-dioleoyl-sn-glycero-3-phosphoethanolami ne-N-[4-(p-maleimidophenyl)butyramide], 1,2-dioleoyl-sn-glycero-3-phosphoethanolamine-N-[3-(2-pyridyldithio)propionate], 1,2-dioleoyl-sn-glycero-3-phosphoethanolamine-N-(biotinyl), 1,2-Dioleoyl-sn-Glycero-3-Phospho(Ethylene Glycol), 1,2-dioleoyl-sn-glycero-3-phosphoethanolamine-N-lactosyl, 1,2-distearoyl-sn-glycero-3-phosphoethanolamine-N-[dibenzocyclooctyl(polyethylene glycol)-2000], 1,2-di stearoyl-sn-glycero-3-phosphoethanolamine-N-[succinyl(polyethylene glycol)-2000], 1,2-distearoyl-sn-glycero-3-phosphoethanolamine-N-[carboxy(polyethylene glycol)-2000], 1,2-distearoyl-sn-glycero-3-phosphoethanolamine-N-[maleimide(polyethylene glycol)-2000], 1,2-distearoyl-sn-glycero-3-phosphoethanolamine-N-[PDP (polyethylene glycol)-2000], 1,2-di stearoyl-sn-glycero-3-phosphoethanol amine-N-[amino(polyethylene glycol)-2000], 1,2-distearoyl-sii-glycero-3-phosphoethanolamine-N-[biotinyl(polyethylene glycol)-2000], 1,2-distearoyl-sn-glycero-3-phosphoethanolamine-N-[cyanur(polyethylene glycol)-2000], 1,2-distearoyl-sn-glycero-3-phosphoethanolamine-N-[folate(polyethylene glycol)-2000], cholesterol modified oligonucleotides, cholesterol-PEG2000-azide, cholesterol-PEG2000-Dibenzocyclooctyl, cholesterol-PEG2000-maleimide, cholesterol-PEG2000-N-hydroxysuccinimide esters, cholesterol-PEG2000-thiol, cholesterol-azide, cholesterol-Dibenzocyclooctyl, cholesterol-maleimide, cholesterol-N-hydroxysuccinimide esters, cholesterol-thiol, C18 modified oligonucleotides, C18-PEG2000-azide, C18-PEG2000-Dibenzocyclooctyl, C18-PEG2000-maleimide, C18-PEG2000-N-hydroxysuccinimide esters, C18-PEG2000-thiol, C18-azide, C8-Dibenzocyclooctyl, C18-maleimide, C18-N-hydroxysuccinimide esters, C18-thiol.

In some embodiments one or more functionalized amphipathic compounds are comprised together with non-functionalized membrane forming lipids in the lipid component of the NLP of the disclosure also comprising one or more lysolipids. In some embodiments functionalized amphipathic compounds can be functionalized membrane forming lipid. In some embodiments, one or more functionalized membrane forming lipids are added or replace the membrane forming lipids in the lipid component of the NLP herein described also comprising one or more lysolipids.

In particular, the ratio between functionalized membrane forming lipid and membrane forming lipids is dependent on the identity of the functionalized membrane forming lipid, and it can be as low as 1% or even lower and as high as 100% as NLPs have been successfully formed with 100% functionalized membrane forming lipid such as DGS-NTA(Ni) (1,2-di-(9Z-octadecenoyl)-sn-glycero-3-[(N-(5-amino-1-carboxypentyl)iminodiacetic acid)succinyl] (nickel salt)). This suggests that NLPs can be formed with any percentage of functionalized membrane forming lipid (from 0 to 100%), depending on the specific functionalized membrane forming lipid used.

In some embodiments, the ratio of functionalized amphipathic compounds can vary from 0.1 mol % to 90 mol % (relative to lysolipids) depending on the functionalized amphipathic compounds. Functionalized amphipathic compounds that are lipids themselves, such as DOGS-NTA-Ni (1,2-di-(9Z-octadecenoyl)-sn-glycero-3-[(N-(5-amino-1-carboxypentyl)iminodiacetic acid)succinyl] (nickel salt)) can be used at 90 mol %, with lysolipids comprising at least 10 mol %. A preferred molar ratio of DOGS-NTA-Ni:lysolipids:membrane forming lipid is 17:50:33. In contrast, functional amphipathic compounds that are less lipid-like, such as cholesterol modified oligonucleotides, a lower mol % (0.1-10 mol %) is needed for successful NLP assembly.

In some embodiments, the lysolipids-incorporated nanolipoprotein particles herein described can further comprise other functional molecules embedded in the membrane lipid bilayer (e.g. interacting with the membrane lipid bilayer components through van der waals forces), conjugated to a lipophilic anchor compound inserted into the membrane lipid bilayer (e.g. through hydrophobic-hydrophilic interactions) or conjugated through binding of a functional group with a corresponding functional group presented on functionalized membrane forming lipid of the membrane lipid bilayer. In some of those embodiments, the other functional molecules comprise small molecules and in particular cyclic or non-cyclic peptides and can be comprised in the NLP here described in an amount that varies from case to case, and that in general can range from 0.1-10 mol %.

In some embodiments, the nanolipoprotein particles herein described can further comprise one or more membrane proteins herein also indicated as target protein. The term “membrane protein” as used herein indicates any protein having a structure that is suitable for attachment to or association with a biological membrane or biomembrane (i.e. an enclosing or separating amphipathic layer that acts as a barrier within or around a cell). In particular, membrane proteins include proteins that contain large regions or structural domains that are hydrophobic (the regions that are embedded in or bound to the membrane); those proteins can be difficult to work with in aqueous systems, since when removed from their normal lipid bilayer environment those proteins tend to aggregate and become insoluble.

Exemplary methods to provide nanolipoprotein particles which are expected to be applicable to provide one or more NLPs presenting one or more membrane proteins, comprise the methods described in U.S. Patent Publication No. 2009/0192299 related to methods and systems for assembling, solubilizing and/or purifying a membrane associated protein in a nanolipoprotein particle, which comprise a temperature transition cycle performed in presence of a detergent, wherein during the temperature transition cycle the nanolipoprotein components are brought to a temperature above and below the gel to liquid crystallization transition temperature of the membrane forming lipid of the nanolipoprotein particle. In some embodiments, verification of inclusion of a membrane proteins can be performed using the methods and systems for monitoring production of a target protein in a nanolipoprotein particle described in U.S. Patent Publication No. 2009/0136937 filed on May 9, 2008 with Ser. No. 12/118,530 which is incorporated by reference in its entirety.

In particular, in several embodiments any one of the nanolipoprotein particle herein described further comprises an active target molecule, such as an immunogen, a drug, a contrast agent or another molecule of interest, comprised as a membrane protein or as an active target molecule attached to functionalized amphipathic compounds in the membrane lipid bilayer, in a configuration resulting having the active target molecule presented on the nanolipoprotein particle. The active target molecule can be a target protein having a hydrophobic region, and be presented on the nanolipoprotein particle attached to the membrane lipid bilayer through interaction of the target protein hydrophobic region with the membrane lipid bilayer. In addition or in the alternative the active target molecule can be an active target molecule presented on the nanolipoprotein particle attached to one or more functionalized membrane forming lipid through anchor compounds as described in U.S. Pat. No. 8,883,729 issued on Nov. 11, 2014 and in U.S. Pat. No. 8,889,623 issued on Nov. 18, 2014 each of which is incorporated by reference in its entirety.

In embodiments herein described NLP comprising lysolipids and optionally additionally components such as polymerizable lipids, amphipathic compounds and/or target proteins and assembled as herein described, are then contacted with a hydrophobically anchorable target molecule or cargo to allow loading of the cargo in the NLP comprising lysophospholipid.

The term “hydrophobically anchorable target molecule” as used herein indicates a molecule comprising an anchor moiety of approximately 2000 Da having a LogK_{ow} lower than 3.5 at conditions compatible with the integrity of the NLP structure, the anchor moiety presented on the molecule for interaction with the lipid component of the NLPs of the disclosure. In hydrophobically anchorable target molecules herein described, K_{ow} defined as the ratio of the molar concentration of the molecule in n-octanol divided by the molar concentration in water of a well-mixed n-octanol water mixture. Conditions compatible with the integrity of the NLP structure typically comprise pH from 5 to 9, and a temperature from 20° C. to 37° C. LogK_{ow} can be measured with several methods including sampling of mixtures of n-octanol and water, high performance liquid chromatography and in silico modeling as well as additional techniques identifiable by a skilled person.

In some embodiments, the hydrophobically anchorable target molecule consists of the anchor moiety and is therefore formed by a molecule of approximately 2000 Da and having a LogK_{ow} lower than 3.5 at conditions compatible with the integrity of the NLP structure. In some embodiments, the anchor moiety is part of a larger molecule where the anchor moiety is presented for interaction with the NLP lipid bilayer. In some of those embodiments the hydrophobically anchorable target molecule can be formed for example by a protein wherein post-translational modification, such as palmitoylation or farnesylation, provide sufficiently hydrophobic “handles” that enable enhanced association with the NLPs when mediated by lysophospholipid. Although such “handles” are naturally occurring in proteins, they can be chemically synthesized and ligated to peptides, RNA and DNA molecules, which can then be incorporated into NLPs.

Following interaction with an NLP herein described for a time and under condition allowing hydrophobic interactions between the hydrophobically anchorable target molecule and the lipid bilayer of the NLP, the hydrophobic interaction results in at least a portion of the anchor moiety of the hydrophobically anchorable target protein being embedded in the lipid bilayer. In particular, in at least some embodiments, the anchor moiety of the hydrophobically anchorable target molecule binding to the lipid bilayer displace at least some of the lysolipids in the lipid bilayer of the NLP as will be understood by a skilled person. In some embodiments, where the hydrophobically anchorable target molecule consist of the anchor moiety, the hydrophobically anchorable target molecule can be entirely embedded in the lipid bilayer, depending on the dimension of the hydrophobically anchorable target molecule and on the presence in the NLP of additional hydrophobically anchorable target molecule and/or other target molecule as will be understood by a skilled person upon reading of the present disclosure.

Exemplary hydrophobically anchorable target molecules thus comprise small molecules, peptides, or proteins which can consist of or comprise an anchor moiety of up to 2,000 Da and with LogK_{ow} lower than 3.5. For example, the drug doxorubicin hydrochloride has a LogK_{ow} of 3.5 at pH 5, 7 or 9, but does not incorporate to a useful degree in NLPs regardless of whether a lysophospholipid is used as part of the drug loading process. In contrast, paclitaxel has a log K_{ow} of 1.27, and is capable of being incorporated into NLP to a useful mass percent when aided by lysophospholipid. The hydrophobically anchorable target molecule can be comprised in the NLP alone or in combination depending on the size of the anchor moiety and the size of the hydrophobically anchorable target molecule as will be understood by a skilled person. It has been demonstrated that drugs up to 900 daltons can be loaded, but larger molecules into the low kilodalton range are expected to also be incorporated using this technique. For comparison, a typical NLP can have a mass up to 600 kilodaltons.

Accordingly, additionally exemplary hydrophobically anchorable target molecules can include antibodies, antibody-drug conjugates, therapeutic proteins, cytokines, DNA, RNA, DNA or RNA modified with hydrophobic tags, PEG or peptide-modified cage molecules for delivery of imaging and radiotherapy isotopes. Cholesterol tagged NOTA or DOTA that may have a targeting ligand attached to bind to tumor-specific cell surface markers and additional molecules identifiable by a skilled person.

One or more hydrophobically anchorable target molecule can be provided in NLPs according to methods comprising contacting the hydrophobically anchorable target molecule with a nanolipoprotein particle to provide a nanolipoprotein particle comprising the hydrophobically anchorable target molecule within a membrane lipid bilayer. In particular, in some embodiments, to assemble lysophospholipid-containing NLPs, a lipid mixture containing lysolipids and membrane forming lipids can be prepared at the desired ratio. NLPs containing lysolipids can be then prepared by using any of the methods described above that are intended for NLP assembly. Once the NLPs are formed, the hydrophobically anchorable cargo (dissolved in a solvent) can then be added to the NLP solution and allowed to mix to allow incorporation of the hydrophobically anchorable cargo (e.g. between 30 minutes and 12 hours). The combined solution can then be treated to remove any remaining insoluble hydrophobically anchorable target molecule that did not incorporate into NLPs (e.g. by centrifugation at approximately 4000 k RPM). The remaining supernatant can then be purified by techniques such as size-exclusion chromatography to remove any remaining dissolved hydrophobically anchorable target molecule that is not incorporated in the particles. Additional variations and embodiments of methods to incorporate a hydrophobically anchorable target molecule in an NLP of the disclosure will be identifiable by a skilled person upon reading of the present disclosure.

In several embodiments, lysolipids-incorporating NLPs herein described can be used in various applications wherein loading with a molecular cargo and particular functionality of NLPs is desired.

In some embodiments, lysolipid-incorporating NLPs herein described can used in biomedical applications, including drug delivery [31-33] [2], diagnostic imaging [34], and vaccine and immunomodulation applications [35-38]. In particular, lysolipid —incorporating NLPs herein described can be used in drug delivery in order to achieve improved pharmacokinetic and drug loading capacity [39, 40]. Nanoparticle-mediated drug delivery performed with lysolipids-incorporating NLPs herein described is expected to address several limitations of conventional drug delivery systems, including nonspecific biodistribution, low water solubility, toxicity due to formulating agents, poor oral bioavailability, and low therapeutic indices [41] [H. Gelderblom, J. Verweij, K. Nooter and A. Sparreboom, European Journal of Cancer, 2001, 37, 1590-1598.].

In particular, in some embodiments, the methods described in this application improve the loading capacity of target drug molecules, particularly hydrophobic drugs.

The term “hydrophobic drug” indicates a group of drug molecules that are hydrophobic and not water-soluble, such as drugs listed in the Biopharmaceutics Classification System (BCS) Class II and Class IV as having low solubility. Exemplary hydrophobic drugs include but not limited to paclitaxel, phenytoin, amphotericin B (AMB), cytosine-arabinoside (ara-C), 5-fluoro-deoxyuridine (5-FdU) and ethinylcytidine. (ETC).

Common administration routes for hydrophobic drugs are limited to oral delivery, local injection, inhalation and surface retention, and intravenous injection if the drug has been formulated appropriately. Intravenous injection is appealing because it has the highest bioavailability (almost 100%) among all administration routes with advantages in immediate effect, targeting effect and overcoming the first pass effect and other advantageous effects known to a skilled person. For example, the tumor targeting effect induced by intravenous administration has become a long-term interest of oncology.

Additional to the administration routes, various formulation strategies need to be adopted in order to enhance the absorption of the hydrophobic drugs. For example, the hydrophobic drug paclitaxel need be formulated in a mixture of ethanol and a castor oil derivative to be given as a free drug. These formulation ingredients are known to be toxic and cause side effects.

Efforts have been made to combine target drug molecules with lipid based carrier systems such as nanoparticles or synthetic versions of nanoparticles found in vivo. However, many synthetic types of nanoparticles have potential disadvantage, such as an inherent toxicity [42] [43], poor control of the size distribution of the particles, physical instability of the product, low drug loading, unsuitability for chronic use and so on.

In some embodiments, the lysolipids-incorporating NLP compositions and methods described in this application can improve the loading capacity of target drug molecules, particularly hydrophobic drugs, by a factor of 20-30 compared to the NLPs assembled in the absence of the lysolipids. The incorporation of lysolipids, within the molar ratios indicated above or understood by a person skilled in the art, can still maintain the desired stability of the NLPs, i.e. without disrupting the planar bilayer structure and the stability of the formed NLPs.

In some embodiments, the hydrophobic drugs can displace the lysolipids from the formed NLPs once the drugs are incorporated and the lysolipids will be expulsed from the assembled NLPs. In other words, the lysolipids of the NLPs act as a placeholder for the drug molecules to be incorporated.

In some embodiments, the lysolipids-incorporating NLP composition can be customized for desired drug loading capacity and stability by adjusting the molar ratio between the molar ratio of the total lipid component and the scaffold protein component and the lysolipids and the membrane forming lipids.

In some embodiments, the lysolipids-incorporating NLPs used for drug loading have a lipid component to scaffold molar ratio ranging from 20:1 to 240:1, depending on the scaffold protein and the lipid component used as will be understood by a skilled person. For example, in NLPs herein described having apoE422k variants as scaffold protein and a lipid component comprising DOPC as the membrane forming lipid and lyso 14:0, the molar ratios of lipid component: scaffold protein component can range from 40:1 to 240:1, preferably from 40:1 to 100:1, even more preferably at 80:1.

In some embodiments, the lysolipids-incorporating NLPs used for drug loading have a lipid component comprising membrane forming lipids in an amount from 90 to 30 mol % of the lipid component and lysolipids in an amount from 10 to 70 mol % of the lipid component, depending on the membrane forming lipids and lysolipids as will be understood by a skilled person. In some embodiments, the NLP herein described comprise membrane forming lipid and lysolipids in molar ratios ranging from 9:1 to 3:7.

For example, in NLPs herein described having a lipid component comprising DOPC as the membrane forming lipid and lyso 14:0 as lysolipids, the DOPC has a mol percentage ranging from 30 mol % to 90 mol %, preferably from 40 mol % to 70 mol % and the lyso 14:0 has a mol percentage ranging from 10 mol % to 70 mol %, preferably from 30 mol % to 60 mol %. In some preferred embodiments, the lyso 14:0 has a 50 mol % (see Example 4).

The desired drug loading capacity of the formed NLP composition can also be achieved by choosing lysolipids with different CMCs (i.e. aqueous solubility), with this feature being dependent on the chain length, the level of saturation of the hydrocarbon chain of the lysolipids, and the structure of the headgroup.

In some embodiments, lysolipids incorporated into the NLPs for improved drug loading have a saturated hydrocarbon chain. Exemplary of lysolipids with saturated hydrocarbon chain include but not limited to 1-tetradecanoyl-sn-glycero-3-phosphocholine (lyso 14:0), 1-hexadecanoyl-sn-glycero-3-phosphocholine (lyso 16:0), 1-octadecanoyl-sn-glycero-3-phosphocholine lyso (18:0).

In other embodiments, lysolipids incorporated into the NLPs for improved drug loading have an unsaturated hydrocarbon chain with one or more double bonds between carbon atoms. Preferably, the unsaturated hydrocarbon chain of the lysophospholipids has a chain length equal to or below 18.

In some embodiments, lysolipids incorporated into the NLPs for drug loading have an aliphatic chain with a chain length between 6 and 18, preferably between 10 and 18, mostly preferably 14. The term “chain length” is characterized by the number of carbons in the aliphatic chain.

In some embodiments, lysolipids incorporated into the NLPs have a CMC value in a range between 0.0005 to 100 mM, preferably between 0.05 to 10 mM, and mostly preferably at about 0.1 mM.

In some embodiments, NLP composition can be customized through apolipoprotein and lipid choice [44, 45] and composition and self-assembly protocols optimized to solubilize membrane proteins,[46-52] protein pore complexes,[53] or hydrophobic drugs [54-56]. Thus, due to the versatility in assembly components, NLPs can be tailor-made for a variety of applications, including targeted drug delivery, antigen delivery,[45, 57] and immune stimulation [45] as will be understood by a skilled person.

In some embodiments, an NLP can be included in pharmaceutical compositions (e.g. a vaccine) together with an excipient or diluent. In particular, in some embodiments, pharmaceutical compositions are disclosed which contain NLP, in combination with one or more compatible and pharmaceutically acceptable vehicle, and in particular with pharmaceutically acceptable diluents or excipients.

The term “excipient” as used herein indicates an inactive substance used as a carrier for the active ingredients of a medication. Suitable excipients for the pharmaceutical compositions herein disclosed include any substance that enhances the ability of the body of an individual to absorb the NLP. Suitable excipients also include any substance that can be used to bulk up formulations with NLP to allow for convenient and accurate dosage. In addition to their use in the single-dosage quantity, excipients can be used in the manufacturing process to aid in the handling of NLP. Depending on the route of administration, and form of medication, different excipients may be used. Exemplary excipients include but are not limited to antiadherents, binders, coatings disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluent includes any substance that can decrease the viscosity of a medicinal preparation.

In certain embodiments, compositions and, in particular, pharmaceutical compositions can be formulated for systemic administration, which includes parenteral administration and more particularly intravenous, intradermic, and intramuscular administration. In some embodiments, compositions and, in particular, pharmaceutical compositions can be formulated for non-parenteral administration and more particularly intranasal, intratracheal, vaginal, oral, and sublingual administration.

Exemplary compositions for parenteral administration include but are not limited to sterile aqueous solutions, injectable solutions or suspensions including NLP. In some embodiments, a composition for parenteral administration can be prepared at the time of use by dissolving a powdered composition, previously prepared in a freeze-dried lyophilized form, in a biologically compatible aqueous liquid (distilled water, physiological solution or other aqueous solution).

The term “lyophilization” (also known as freeze-drying or cryodesiccation) indicates a dehydration process typically used to preserve a perishable material or make the material more convenient for transport. Freeze-drying works by freezing the material and then reducing the surrounding pressure and adding enough heat to allow the frozen water in the material to sublime directly from the solid phase to gas.

If a freeze-dried substance is sealed to prevent the reabsorption of moisture, the substance may be stored at room temperature without refrigeration, and be protected against spoilage for many years. Preservation is possible because the greatly reduced water content inhibits the action of microorganisms and enzymes that would normally spoil or degrade the substance.

Lyophilization can also cause less damage to the substance than other dehydration methods using higher temperatures. Freeze-drying does not usually cause shrinkage or toughening of the material being dried. In addition, flavours and smells generally remain unchanged, making the process popular for preserving food. However, water is not the only chemical capable of sublimation, and the loss of other volatile compounds such as acetic acid (vinegar) and alcohols can yield undesirable results.

Freeze-dried products can be rehydrated (reconstituted) much more quickly and easily because the process leaves microscopic pores. The pores are created by the ice crystals that sublimate, leaving gaps or pores in their place. This is especially important when it comes to pharmaceutical uses. Lyophilization can also be used to increase the shelf life of some pharmaceuticals for many years.

In pharmaceutical applications freeze-drying is often used to increase the shelf life of products, such as vaccines and other injectables. By removing the water from the material and sealing the material in a vial, the material can be easily stored, shipped, and later reconstituted to its original form for injection

In some embodiments, lysolipids-incorporating NLPs herein described and related components can be provided as a part of systems in accordance to various embodiments herein described.

In some embodiments, the systems herein described can be provided in the form of kits of parts. In a kit of parts, membrane forming lipid and lysolipids can be provided in various combinations with one or more functionalized amphipathic compounds, one or more membrane protein, and/or scaffold proteins or fragments thereof. In the kits of parts, the components can be comprised in the kit independently possibly included in a composition together with suitable vehicle carrier or auxiliary agents.

Additional components can also be included and comprise, reference standards, and additional components identifiable by a skilled person upon reading of the present disclosure.

In the kit of parts herein disclosed, the components of the kit can be provided, with suitable instructions and other necessary reagents, in order to perform the methods here disclosed. In some embodiments, the kit can contain the compositions in separate containers. Instructions, for example written or audio instructions, on paper or electronic support such as tapes or CD-ROMs, for carrying out the methods herein described, can also be included in the kit. The kit can also contain, depending on the particular method used, other packaged reagents and materials (i.e. wash buffers and the like).

Further details concerning the identification of the suitable carrier agent or auxiliary agent of the compositions, and generally manufacturing and packaging of the kit, can be identified by the person skilled in the art upon reading of the present disclosure.

### EXAMPLES

The methods and system herein disclosed are further illustrated in the following examples, which are provided by way of illustration and are not intended to be limiting.

In particular, NLPs comprising various membrane forming lipids, DiynePC and lysophospholipids were prepared and characterized in vitro. A skilled person will be able to use other membrane forming lipids, lysolipids, scaffold proteins and hydrophobically anchorable target molecules herein described. The following materials and methods were used

Materials:

1,2-dioleoyl-sn-glycero-3-phosphocholine (DOPC), 1,2-bis(10,12-tricosadiynoyl)-sn-glycero-3-phosphocholine (DiynePC), 1-tetradecanoyl-sn-glycero-3-phosphocholine (lyso 14:0), 1-hexadecanoyl-sn-glycero-3-phosphocholine (lyso 16:0), 1-octadecanoyl-sn-glycero-3-phosphocholine lyso (18:0), and 1-(9Z-octadecenoyl)-sn-glycero-3-phosphocholine (lyso 18:1) were purchased from Avanti Polar Lipids (Alabaster, Ala.). All other reagents were ordered from Sigma-Aldrich (St. Louis, Mo.).

Protein Expression and Purification:

The expression clone for the 22 kDa N-terminal fragment of human apolipoprotein E4 (apoE422k, kindly provided by Dr. Karl Weisgraber) featuring a cleavable His-tag[58] was expressed and purified as previously described.[6, 59].

Preparation of Paclitaxel:

Paclitaxel was obtained from LC Laboratories (Woburn, Mass.) and was prepared in solutions of dimethylformamide or dimethylsulfoxide at a concentration of 50 mg/ml.

### Example 1 LysoNLP Assemblies and Purification

NLPs comprising lysolipids were assembled according to a previously reported procedure [6, 59] with slight modifications.

For NLP assemblies using DOPC as the membrane forming lipid with apoE422k, the total lipid-to-apoE422K molar ratio was 80:1. Briefly, lipids were either prepared or obtained in chloroform and aliquoted into glass vials. Chloroform was then removed using a stream of N₂ under agitation to form a thin lipid film. Lipids were solubilized in PBS buffer (137 mM sodium chloride, 2.7 mM potassium chloride, 10 mM phosphate buffer, pH 7.4) using 30 mM sodium cholate. After addition of the apoE422k (150 μM in final assembly volume), samples were incubated at 23.8° C. for at least 2 hours.

Assemblies were dialyzed overnight against PBS to remove cholate. Samples were subsequently analyzed and purified by SEC (size exclusion chromatography) (Superdex 200, 10/300 GL column, GE Healthcare, Piscataway, N.J.) in PBS buffer (0.5 mL/min flow rate). The exclusion limit of the column was determined with Blue Dextran 2000. SEC fractions (500 μl) were collected every 60 s. SEC fractions containing homogeneous NLP populations were concentrated using 50 kDa MWCO spin concentrators (Sartorius). The apoE422k concentration was determined using NanoDrop (Thermo Scientific). The concentrated NLP samples were then stored at 4° C. until further use.

NLP samples were subsequently analyzed and purified by SEC (Superdex 200, 10/300 GL column, GE Healthcare, Piscataway, N.J.) in PBS buffer (0.5 mL/min flow rate). The exclusion limit of the column was determined with Blue Dextran 2000. SEC fractions (500 μl) were collected every 60 s. SEC fractions containing homogeneous NLP populations were concentrated using 50 kDa MWCO spin concentrators (Sartorius). A concentration for NLPs in solution was determined by using a Nanodrop ND-1000 spectrophotometer (ThermoScientific, Lafayette, Colo.) at an absorbance of 280 nm.

The concentrated NLP samples were then stored at 4° C. until further use. In these experiments, the NLP concentration was calculated based on the apoE422k concentration by assuming that each NLP contained 6 apoE422k scaffold proteins.[3, 59]

### Example 2: Preparation of NLP Mixture Loaded with Paclitaxel

A desired amount of paclitaxel was added to the purified NLP solution of Example 1 and allowed to mix overnight. The solution was then spun down to remove any precipitate, and then the supernatant was purified by SEC (GE HiTrap desalting column) in PBS buffer (0.5 ml/min flow rate). The collected fractions containing NLP were then analyzed by reverse phase HPLC to quantify each component of the NLP.

### Example 3: Characterization of NLPs Formulated with Lysophospholipids

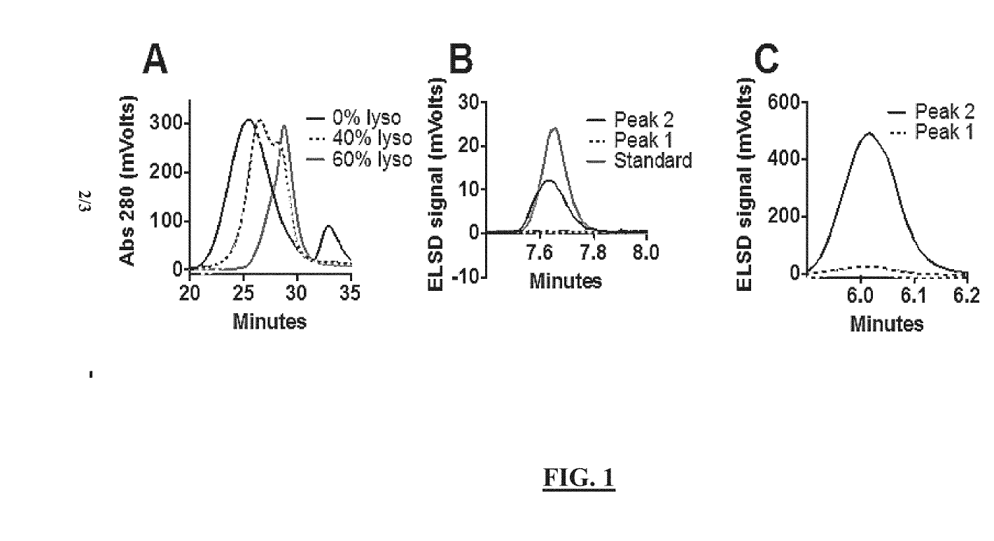

Purified NLPs were then analyzed by size exclusion chromatography (SEC). FIG. 1, panels A-C show the characterization of NLPs formed with lysophospholipids (lyso 14:0) subsequently loaded with paclitaxel. FIG. 1, panel A is a plot showing representative SEC traces of NLPs prepared with increasing mol % (0 mol %, 40 mol % and 60 mol %) of lysophospholipid. FIG. 1, panel B is a plot showing representative RP-HPLC traces of purified fractions from SEC of the 40 mol % NLPs depicted in FIG. 1, panel A. FIG. 1, panel C plots representative RP-HPLC traces showing the differences in paclitaxel loaded into the two NLP populations.

NLPs prepared with lysophospholipids at 40 mol % eluted as two populations that were centered at 26 and 27 minutes, respectively, as evidenced by the double peaks in FIG. 1, panel A. This suggests a difference in NLP size between the two populations, with the larger particles eluting at early time (i.e. 26-munte) and the smaller particles 7 eluting at later time (i.e. 27-minute). These populations both absorb at 280 nanometers, consistent with the presence of the apolipoprotein scaffold on the particle. As the lysophospholipid content was modulated, the relative intensity of these two peaks modulated accordingly, with lower lysophospholipid contents (0 mol %) producing a larger first peak, and higher lysophospholipid contents (60 mol %) yielding a larger second peak.

Representative fractions from these two peaks from NLPs with an initial lysophospholipid content of 40 mol % were collected and then analyzed by reverse phase high performance liquid chromatography (RP-HPLC) in order to compare the relative ratios of components found in each sample (FIG. 1, panel B). As shown in FIG. 1, panel B, compositional differences were observed between the two populations of NLPs. The largest compositional difference was the lysophospholipid content of the NLPs, with the second peak having much greater quantities of the single-chained lipids (i.e. lysophospholipid). This result indicates that the NLPs formed with lysophospholipids differ not only in size, but also in composition.

The ability of the two NLP populations to accommodate the drug of interest, paclitaxel, was then assessed. Following addition of paclitaxel to the two solutions, the NLP solutions were re-purified to remove free paclitaxel. The solutions were then analyzed by reverse phase HPLC to assess the amount of paclitaxel that was incorporated into the particles. A representative example of the differences in paclitaxel signal between the first and second peaks is shown in FIG. 1, panel C. The NLPs from the second peak, shown in solid line, appear to accept substantially more of the drug than the larger particles from peak 1, shown in dotted line. Also, the intensity of the peak corresponding to the lysophospholipid is reduced upon addition of the paclitaxel and subsequent purification.

Based on the above results, one of the findings is that when NLPs are formed with lysophospholipids, the whole population of particles appears to be more heterogeneous in size, whereby two distinct populations can be identified (as shown in FIG. 1, panel A, dotted line). The NLPs particles in the first population are more similar in properties to NLPs not formulated with lysophospholipids. These particles are approximately the same size as lysophospholipid-free NLPs, contain relatively small amount of lysophospholipid, and are only able to accommodate a small amount of the hydrophobic drug paclitaxel. In contrast, the NLPs particles in the second population are substantially smaller in size than those in the first population. These particles appear to be rich in lysophospholipids, and are a suitable acceptor of paclitaxel.

It is also found that the lysophospholipid content of these NLPs particles diminishes with the addition of paclitaxel and re-purification of the sample. This suggests that the paclitaxel can displace the lysophospholipids from the particle upon incorporation, and that these lysophospholipids can be separated out by SEC. It has been previously shown that the expulsion of lysolipids from a lipid bilayer creates defects in lipid bilayers,[60] and it is expected that these defects can be used under the appropriate conditions, as insertion points for the paclitaxel.

Another finding from the above results is that NLP populations have been observed to form multiple populations leading to high degree of compositional heterogeneity even when prepared with single lipid species and such NLP populations differ widely in the same sample.

### Example 4: Assessment of the Relationship Between Paclitaxel Loading and Lysophospholipid Content (Lyso 14:0)

Further experiments were carried out to assess the relationship between lysophospholipid content and paclitaxel loading. NLPs were prepared with initial lysophospholipid contents (lyso 14:0) between 0 and 70 mole percent (i.e. 0 mol %-70 mol %). After paclitaxel addition and subsequent purification, the composition of these particles was again analyzed using reverse phase HPLC.

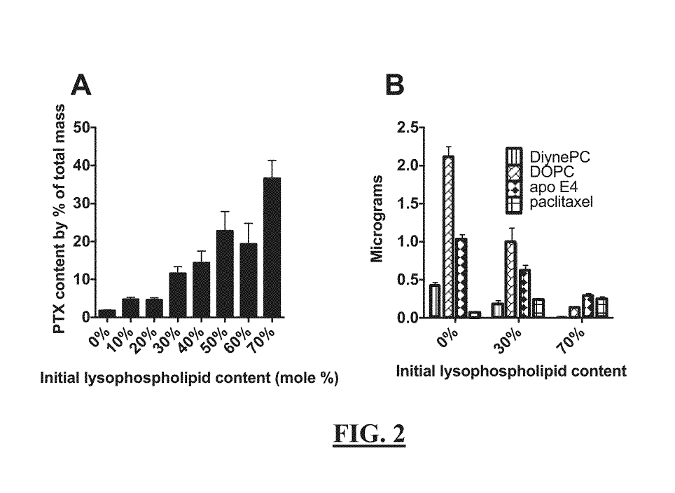

FIG. 2, panel A is a plot showing the amount of paclitaxel loaded into NLPs (mass of drug over total mass of particle components) as a function of initial lysophospholipid content (mol %). Consistent with the previous results shown in Example 3, FIG. 1, panel C, FIG. 2, panel A shows that the paclitaxel content in NLPs (y-axis) without lysophospholipids appears to be 1-2% by mass of the particle. However, once lysophospholipids are added to the particle, the amount of paclitaxel in the particle increases relative to the other components. For 10-20 mol % lysophospholipid, the paclitaxel content is approximately 5% by mass. For lysophospholipid contents ranging from 30-60 mol⁰/o, the paclitaxel loading ranges from approximately 10-20%. At 70 mol % lysophospholipid content, the paclitaxel loading is in excess of 30% by mass.

FIG. 2, panel B is a plot showing the amounts of each NLP component (at y-axis) measured for NLPs prepared with different initial lysophospholipid contents at 0 mol %, 30 mol % and 70 mol %. The plot shows that the measured quantity of paclitaxel is nearly the same between the 30 mol % and 70 mol % lysophospholipid groups (i.e. about 0.23 micrograms). However, the amount of other NLP components appears to diminish. The results suggest that while these NLPs can accept higher amounts of paclitaxel, a higher amount of paclitaxel would result in more unstable particles with lower yields after the SEC purification.

### Example 5: Assessment of the Relationship Between Paclitaxel Loading and Lysophospholipid Content (Lyso 16:0, Lyso 18:0 and Lyso 18:1)

To further examine the relationship between lysophospholipids and paclitaxel loading, several other lysophospholipid species were incorporated into NLPs and then loaded with paclitaxel. While the first lysophospholipid tested in Examples 4 (lyso 14:0) had a saturated chain with a length of fourteen carbons, the others tested in this example were lyso 16:0 and lyso 18:0 both having a saturated chain with a length of sixteen and eighteen carbons, respectively, and lyso 18:1 having an unsaturated chain.

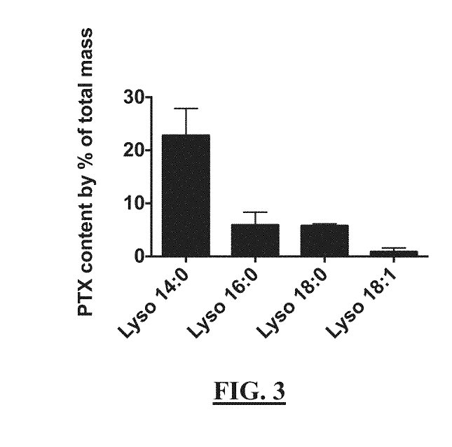

FIG. 3 is a plot showing the amount of paclitaxel loaded into NLPs (at y-axis) as a function of lysophospholipid acyl chain structure (at x-axis). The y-axis is calculated as the mass of drug over total mass of particle components. The lysophospholipids of the x-axis are prepared at 50 mol % content.

As shown in FIG. 3, lyso 14:0 yields particles capable of accepting the highest amount of paclitaxel (over 20%), while lyso 18:1 yields particles accepting the least amount of paclitaxel, The results indicate the correlation between the paclitaxel loading and the chain length of lysophospholipids, That is, lysophospholipids with shorter chain-length can yield NLPs particles with higher paclitaxel loading capacity.

It has been demonstrated by others that lysophospholipids with shorter chain-length can affect the planar biplayer structure and the stability of the formed NLPs. [ref. 19]. Accordingly the chain length of the lysophospholipids is selected for enhanced drug loading capacity with minimized perturbation to the structure and stability of the NLP particles.

In addition, the paclitaxel loading also correlates to the critical micelle concentration (CMC), or aqueous solubility of the lysophospholipids. Lyso 18:1 has the lowest solubility (or lack of a CMC); lyso 16:0 and 18:0 have intermediate CMCs at ˜6 and 0.4 μM, respectively; and lyso 14:0 has the highest CMC value of ˜80 μM. These results suggest that more soluble lysophospholipids are better suited to formulate NLPs capable of accepting paclitaxel. In the scenarios when paclitaxel displaces the lysophospholipids, such displacement can be more easily achieved with lysophospholipids that are more soluble in the surrounding aqueous environment.

Results from the above examples indicate that with the incorporation of lysophospholipids, higher quantities of paclitaxel can be loaded into the pre-formed NLP particles. It has also been demonstrated in the above examples that the paclitaxel loading capacity of lysoNLPs can be affected by both the solubility and the chain length of the lysophospholipids. Other factors are expected to also play a role in drug loading, such as the head group of the lysophospholipids.

In summary, described herein are nanolipoprotein particles comprising at least a scaffold protein component and a membrane lipid component and related compositions, methods and systems are described, in which the membrane lipid component comprises at least one or more membrane forming lipids and one or more lysoplipids.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure. Those skilled in the art will recognize how to adapt the features of the exemplified NLPs and related uses to additional NLPs formed by other lysophospholipids, membrane forming lipids, scaffold proteins and possibly functionalized amphipathic compounds and membrane proteins according to various embodiments and scope of the claims.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

### REFERENCES

1. Kreshech, G. C., Surfactants in Water—A Comprehensive Treatise. 1975: Plenum, N.Y.
2. Fischer, N. O., et al., Evaluation of Nanolipoprotein Particles (NLPs) as an In Vivo Delivery Platform. PLoS ONE, 2014, 9(3): p. e93342.
3. Blanchette, C. D., et al., Kinetic Analysis of His-Tagged Protein Binding to Nickel—Chelating Nanolipoprotein Particles. Bioconjugate Chemistry, 2010. 21(7): p. 1321-1330.
4. Fischer, N. O., et al., Colocalized Delivery of Adjuvant and Antigen Using Nanolipoprotein Particles Enhances the Immune Response to Recombinant Antigens. Journal of the American Chemical Society, 2013. 135(6): p. 2044-2047.
5. Shih, A. Y., et al., Disassembly of nanodiscs with cholate. Nano Letters, 2007. 7(6): p. 1692-1696.
6. Chromy, B. A., et al., Different apolipoproteins impact nanolipoprotein particle formation. Journal of the American Chemical Society, 2007. 129(46): p. 14348-14354.
7. Blanchette, C. D., et al., Characterization and Purification of Polydisperse Reconstituted Lipoproteins and Nanolipoprotein Particles. International Journal of Molecular Sciences, 2009. 10(7): p. 2958-2971.
8. CHROMY, B. A., NANOLIPOPROTEIN PARTICLES AND RELATED METHODS AND SYSTEMS FOR PROTEIN CAPTURE, SOLUBILIZATION, AND/OR PURIFICATION in US 2009-0192299 A1 2009: US.
9. Cappuccio, J. A., et al., Cell-free Co-expression of Functional Membrane Proteins and Apolipoprotein, Forming Soluble Nanolipoprotein Particles. Molecular & Cellular Proteomics, 2008. 7(11): p. 2246-2253.
10. Gao, T. J., et al., Characterizing diffusion dynamics of a membrane protein associated with nanolipoproteins using fluorescence correlation spectroscopy.

Protein Science, 2011. 20(2): p. 437-447.
11. Katzen, F., et al., Insertion of membrane proteins into discoidal membranes using a cell-free protein expression approach. Journal of Proteome Research, 2008. 7(8): p. 3535-3542.
12. Coleman, M. A., METHODS AND SYSTEMS FOR PRODUCING NANOLIPOPROIEIN PARIICLES, in US 2011-0059549 A1 2008: US.
13. Georger, J. H., et al., Helical and Tubular Microstructures Formed by Polymerizable Phosphatidylcholines. J Am Chem Soc, 1987. 109(20): p. 6169-6175.
14. Morigaki, K., et al., Surface Functionalization of a Polymeric Lipid Bilayer for Coupling a Model Biological Membrane with Molecules, Cells, and Microstructures. Langmuir, 2013. 29(8): p. 2722-2730.
15. Regen, S. L., et al., Polymerized Phosphatidyl Choline Vesicles—Stabilized and Controllable Time-Release Carriers. Biochemical and Biophysical Research Communications, 1981. 101(1): p. 131-136.
16. Bolikal, D. and S. L. Regen, Degree of Polymerization of a Vesicle Membrane. Macromolecules, 1984. 17(6): p. 1287-1289.
17. Lei, J. T. and D. F. Obrien, 2-Dimensional Polymerization of Lipid Bilayers—Rate of Polymerization of Acryloyl and Methacryloyl Lipids. Macromolecules, 1994. 27(6): p. 1381-1388.
18. Sells, T. D. and D. F. Obrien, 2-Dimensional Polymerization of Lipid Bilayers—Degree of Polymerization of Acryloyl Lipids. Macromolecules, 1994. 27(1): p. 226-233.
19. Lamparski, H. and D. F. Obrien, 2-Dimensional Polymerization of Lipid Bilayers—Degree of Polymerization of Sorbyl Lipids. Macromolecules, 1995. 28(6): p. 1786-1794.
20. Tsuchida, E., et al., Polymerization of Unsaturated Phospholipids as Large Unilamellar Liposomes at Low-Temperature. Macromolecules, 1992. 25(1): p. 207-212.
21. Ohno, H., Y. Ogata, and E. Tsuchida, Polymerization of Liposomes Composed of Diene-Containing Lipids by Uv and Radical Initiators—Evidence for the Different Chemical Environment of Diene Groups on 1-Acyl and 2-Acyl Chains. Macromolecules, 1987. 20(5): p. 929-933.
22. Serrano, J., et al., Polymerized Surfactant Vesicles—Determinations of Rates and Degrees of Polymerization in Vesicles Prepared from Styrene-Containing Surfactants. Macromolecules, 1985. 18(10): p. 1999-2005.
23. Lieser, G., B. Tieke, and G. Wegner, Structure. Phase-Transitions and Polymerizability of Multilayers of Some Diacetylene Monocarboxylic Acids. Thin Solid Films, 1980. 68(1): p. 77-90.
24. Johnston, D. S., et al., Phospholipid polymers—synthesis and spectral characteristics. Biochim Biophys Acta, 1980. 602(1): p. 57-69.
25. Kim, J. M., et al., Immobilized polydiacetylene vesicles on solid substrates for use as chemosensors. Advanced Materials, 2003. 15(13): p. 1118-1121.
26. Hayward, J. A. and D. Chapman, Biomembrane surfaces as models for polymer design: the potential for haemocompatibility. Biomaterials, 1984. 5(3): p. 135-42.
27. Regen, S. L., et al., Polymerized phosphatidyl choline vesicles. Stabilized and controllable time-release carriers. Biochem Biophys Res Commun, 1981. 101(1): p. 131-6.
28. Yavlovich, A., et al., A novel class of photo-triggerable liposomes containing DPPC:DC8.9PC as vehicles for delivery of doxorubcin to cells. Biochimica Et Biophysica Acta-Biomembranes, 2011. 1808(1): p. 117-126.
29. Tieke, B., G. Lieser, and G. Wegner, Polymerization of diacetylenes in multilayers. Journal of Polymer Science: Polymer Chemistry Edition, 1979. 17(6): p. 1631-1644.
30. Baughman, R. H., Solid-state polymerization of diacetylenes. Journal of Applied Physics, 1972. 43(11): p. 4362-4370.
31. Tufteland, M., G. Ren, and R. Ryan, Nanodisks derived from amphotericin B lipid complex. Journal of Pharmaceutical Sciences, 2008. 97(10): p. 4425-4432.
32. Yuan, Y., et al., Delivery of hydrophilic drug doxorubicin hydrochloride-targeted liver using apoA1 as carrier. J Drug Target, 2013. 21(4): p. 367-374.
33. Ding, Y., et al., A biomimetic nanovector-mediated targeted cholesterol—conjugated siRNA delivery for tumor gene therapy. Biomaterials, 2012. 33(34): p. 8893-8905.
34. Frias, J. C., et al., Properties of a versatile nanoparticle platform contrast agent to image and characterize atherosclerotic plaques by magnetic resonance imaging. Nano Lett, 2006. 6(10): p. 2220-2224.
35. Fischer, N. O., et al., Conjugation to nickel-chelating nanolipoprotein particles increases the potency and efficacy of subunit vaccines to prevent West Nile encephalitis. Bioconjug Chem, 2010. 21(6): p. 1018-22.
36. Fischer, N. O., et al., Colocalized delivery of adjuvant and antigen using nanolipoprotein particles enhances the immune response to recombinant artigens. J Am Chem Soc, 2013. 135(6): p. 2044-7.
37. Weilhammer, D. R., et al., The use of nanolipoprotein particles to enhance the immunostimulatory properties of innate immune agonists against lethal influenza challenge. Biomaterials, 2013. 34(38): p. 10305-18.
38. Bhattacharya, P., et al., Nanodisc-Incorporated Hemagglutinin Provides Protective Immunity against Influenza Virus Infection. Journal of Virology, 2010. 84(1): p. 361-371.
39. Allen, T. M. and P. R. Cullis, Drug delivery systems: entering the mainstream. Science, 2004. 303(5665): p. 1818-22.
40. Sparreboom, A., et al., Comparative preclinical and clinical pharmacokinetics of a cremophor-free, nanoparticle albumin-bound paclitaxel (ABI-007) and paclitarel formulated in Cremophor (Taxol). Clin Cancer Res, 2005. 11(11): p. 4136-43.
41. Cho, K., et al., Therapeutic nanoparticles for drug delivery in cancer. Clinical cancer research, 2008. 14(5): p. 1310-1316.
42. al., A.G.C.e., Emerging implications of nanotechnology on cancer diagnostics and therapeutics. Cancer, 2006. 107: p. 8.
43. al., S.M.e., A concise review of carbon nanotube's toxicology. Nano Rev., 2013. 4.
44. Miyazaki, M., et al., Effect of phospholipid composition on discoidal HDL formation. Biochimica et Biophysica Acta (BBA)-Biomembranes, 2013. 1828(5): p. 1340-1346.
45. Weilhammer, D. R., et al., The use of nanolipoprotein particles to enhance the immunostimulatory properties of innate immune agonists against lethal influenza challenge. Biomaterials, 2013. 34(38): p. 10305-10318.
46. Cappuccio, J. A., et al., Cell-free co-expression of functional membrane proteins and apolipoprotein, forming soluble nanolipoprotein particles. Molecular & Cellular Proteomics, 2008. 7(11): p. 2246-2253.
47. Cappuccio, J. A., et al., Cell-free expression for nanolipoprotein particles: building a high-throughput membrane protein solubility platform, in High throughput protein expression and purification. 2009, Springer. p. 273-295.
48. Blanchette, C. D., et al., Atomic force microscopy differentiates discrete size distributions between membrane protein containing and empty nanolipoprotein particles. Biochimica et Biophysica Acta (BBA)-Biomembranes, 2009. 1788(3): p. 724-731.
49. Wadsater, M., et al., Monitoring shifts in the conformation equilibrium of the membrane protein cytochrome P450 reductase (POR) in nanodiscs. Journal of Biological Chemistry, 2012. 287(41): p. 34596-34603.
50. Justesen, B. H., et al., Isolation of monodisperse nanodisc-reconstituted membrane proteins using free flow electrophoresis. Analytical chemistry, 2013. 85(7): p. 3497-3500.
51. Baylon, J. L., et al., Characterizing the membrane-bound state of cytochrome P450 3A4: structure, depth of insertion, and orientation. Journal of the American Chemical Society, 2013. 135(23): p. 8542-8551.
52. Gao, T., et al., Characterization of De Novo Synthesized GPCRs Supported in Nanolipoprotein Discs. PLoS ONE, 2012. 7(9): p. e44911.
53. Akkaladevi, N., et al., Assembly of anthrax toxin pore: Lethal-factor complexes into lipid nanodiscs. Protein Science, 2013. 22(4): p. 492-501.
54. Tufteland, M., et al., Peptide stabilized amphotericin B nanodisks. Peptides, 2007. 28(4): p. 741-746.
55. Jia, J., et al., Preparation, characterizations, and in vitro metabolic processes of paclitaxel-loaded discoidal recombinant high-density lipoproteins. Journal of Pharmaceutical Sciences, 2012. 101(8): p. 2900-2908.
56. Wang, J., et al., Tumor targeting effects of a novel modified paclitaxel-loaded discoidal mimic high density lipoproteins. Drug delivery, 2013. 20(8): p. 356-363.
57. Fischer, N. O., et al., Conjugation to Nickel-Chelating Nanolipoprotein Particles hIcreases the Potency and Efficacy of Subunit Vaccines to Prevent West Nile Encephalitis. Bioconjugate Chemistry, 2010. 21(6): p. 1018-1022.
58. Rensen, P. C. N., et al., Recombinant lipoproteins: lipoprotein-like lipid particles for drug targeting. Advanced Drug Delivery Reviews, 2001. 47(2-3): p. 251-276.
59. Blanchette, C. D., et al., Quantifying size distributions of nanolipoprotein particles with single-particle analysis and molecular dynamic simulations. Journal of Lipid Research, 2008. 49(7): p. 1420-1430.
60. Howland, M. C. and A. N. Parikh, Model Studies of Membrane Disruption by Photogenerated Oxidative Assault. The Journal of Physical Chemistry B, 2010. 114(19): p. 6377-6385.

1. A nanolipoprotein particle comprising:
a membrane forming lipid, a lysolipid and a scaffold protein,
the membrane forming lipid and the lysolipid arranged in a membrane forming lipid bilayer stabilized by the scaffold protein.

2. The nanolipoprotein particle of claim 1, wherein the lysolipid is in a molar concentration of about 10 to about 70 mol %.

3. The nanolipoprotein particle of claim 1, wherein the lysolipid is in molar concentration of at least 30 mol %.

4. The nanolipoprotein particle of claim 1, wherein a total lipid to scaffold protein molar percent ratio ranges from 20:1 to 240:1.

5. The nanolipoprotein particle of claim 4, wherein a total lipid to scaffold protein molar percent ratio is 80:1.

6. The nanolipoprotein particle of claim 1, wherein the membrane forming lipid is in amount from 20 to 90% and the lysolipid is in an amount from 10 to 80% with respect to a total lipid concentration.

7. The nanolipoprotein particle of claim 1, wherein the lysolipid comprise lysophospholipids and/or lysosphingolipids.

8. The nanolipoprotein particle of claim 1, wherein the lysolipid comprise lipids of Formula (I) 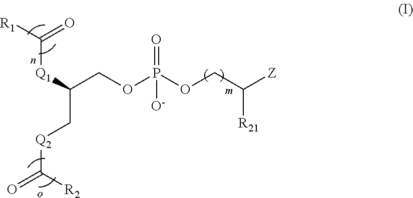
wherein R1 and R2 are independently selected from H, C1-C6 branched or linear substituted or unsubstituted aliphatic chain and a C7-C29 branched or linear, substituted or unsubstituted aliphatic chain; 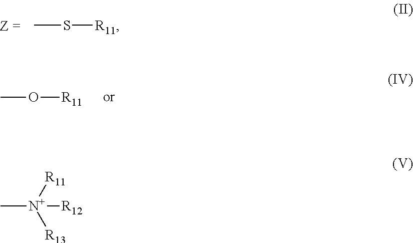
in which R₁₁, R₁₂ and R₁₃ are independently H or a C1-C4 branched or straight aliphatic chain;
R₂₁ is H, OH, or a carboxy group;
Q₁ and Q₂ are independently O, S, CH₂, NH, or NR₁₁;
m=0-3;
one of n and o is 0, and the other one of n and o is 1; and
wherein one of R1 and R2 is the C7-C29 branched or straight, substituted or unsubstituted aliphatic chain and the other one of R1 and R2 is either H or the C1-C6 branched or linear substituted or unsubstituted aliphatic chain.

9. The nanolipoprotein particle of claim 1, wherein the lysolipid comprise lipids of Formula (Ia) 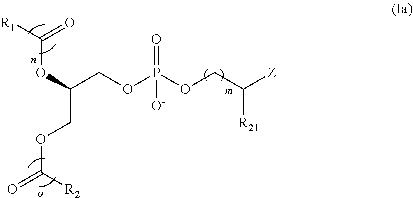
wherein R1 and R2 are independently selected from H, C1-C6 branched or linear, substituted or unsubstituted aliphatic chain and a C7-C29 branched or linear, substituted or unsubstituted aliphatic chain; 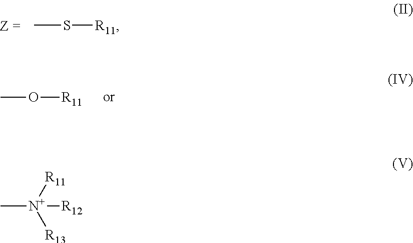
in which R₁₁, R₁₂ and R₁₃ are independently H or a C1-C4 branched or linear aliphatic chain;
R₂₁ is H, OH, or a carboxy group
m=0-3;
one of n and o is 0, and the other one of n and o is 1; and
wherein one of R1 and R2 is the C7-C29 branched or straight, substituted or unsubstituted aliphatic chain and the other one of R1 and R2 is either H or the C1-C6 branched or linear, substituted or unsubstituted aliphatic chain.

10. The nanolipoprotein particle of claim 1, wherein the lysolipid is selected from a group consisting of:
1-tetradecanoyl-sn-glycero-3-phosphocholine (lyso 14:0), 1-hexadecanoyl-sn-glycero-3-phosphocholine (lyso 16:0), 1-octadecanoyl-sn-glycero-3-phosphocholine lyso (18:0) and 1-(9Z-octadecenoyl)-sn-glycero-3-phosphocholine (lyso 18:1).

11. The nanolipoprotein particle of claim 1, wherein the lysolipid has a CMC value ranging between 0.05 to 100 milliMolar.

12. The nanolipoprotein particle of claim 1, wherein the lysolipid has a CMC value of 80 μM.

13. The nanolipoprotein particle of claim 1, wherein the nanolipoprotein particle further comprises one or more functionalized amphipathic compound.

14. A method of loading a hydrophobically anchorable target molecule, the method comprising:
contacting the hydrophobically anchorable target molecule with a plurality of nanolipoprotein particles of claim 1 to provide nanolipoprotein particles loaded with the hydrophobically anchorable target molecule, to allow hydrophobic interactions between the hydrophobically anchorable target molecule and the lipid bilayer of the nanolipoprotein particles and displacement of at least a portion of the one or more lysolipid by the hydrophobically anchorable target molecule.

15. The method of claim 14, further comprising before contacting the hydrophobically anchorable target molecule with the plurality of nanoparticles contacting one or more membrane forming lipids and one or more lysolipid with one or more scaffold proteins to provide the plurality of nanolipoprotein particles in which the one or more lysolipid are comprised within a membrane forming lipid bilayer stabilized by the one or more scaffold proteins.

16. The method of claim 14, wherein the one or more lysolipid are in a molar concentration of about 10 to about 70 mol %.

17. The method of claim 14, wherein the one or more lysolipid are in molar concentration of at least 30 mol %.

18. The method of claim 14, wherein a molar percent ratio between a total lipid comprising the one or more membrane forming lipids and the one or more lysolipid and the scaffold protein ranges from 20:1 to 240:1

19. The method of claim 18, wherein the molar percent ratio is 80:1.

20. The method of claim 14, wherein the membrane forming lipid is in amount from 20 to 90% and the lysolipid is in an amount from 10 to 80% with respect to a total lipid concentration.

21. The method of claim 20, wherein the lysolipid is in an amount from 30 to 60% with respect to a total lipid concentration.

22. The method of claim 14, wherein the hydrophobically anchorable target molecule is a hydrophobic drug.

23. The method of claim 14, wherein the lysolipid comprise lipids of Formula (I) 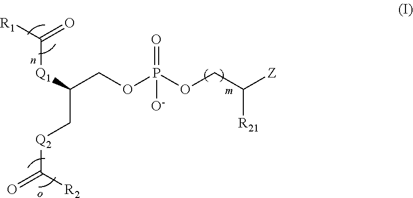
wherein R1 and R2 are independently selected from H, C1-C6 branched or linear substituted or unsubstituted aliphatic chain and a C7-C29 branched or linear, substituted or unsubstituted aliphatic chain; 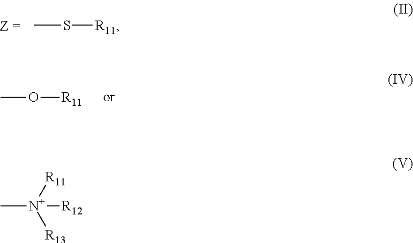
in which R₁₁, R₁₂ and R₁₃ are independently H or a C1-C4 branched or linear aliphatic chain;
R₂₁ is H, OH, or a carboxy group;
Q₁ and Q₂ are independently O, S, CH₂, NH, or NR₁₁;
m=0-3;
one of n and o is 0, and the other one of n and o is 1, and
wherein one of R1 and R2 is the C7-C29 branched or linear, substituted or unsubstituted aliphatic chain and the other one of R1 and R2 is either H or the C1-C6 branched or linear substituted or unsubstituted aliphatic chain.

24. The method of claim 14, wherein the lysolipid comprise lipids of Formula (Ia) 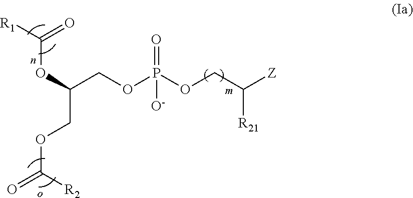
wherein R1 and R2 are independently selected from H, C1-C6 branched or linear, substituted or unsubstituted aliphatic chain and a C7-C29 branched or linear, substituted or unsubstituted aliphatic chain; 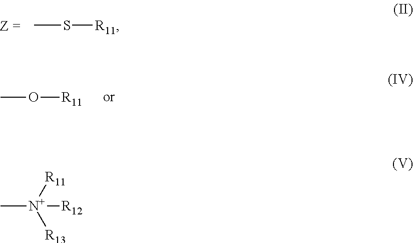
in which R₁₁, R₁₂ and R₁₃ are independently H or a C1-C4 branched or linear aliphatic chain;
R₂₁ is H, OH, or a carboxy group
m=0-3;
one of n and o is 0, and the other one of n and o is 1; and
wherein one of R1 and R2 is the C7-C29 branched or straight, substituted or unsubstituted aliphatic chain and the other one of R1 and R2 is either H or the C1-C6 branched or linear, substituted or unsubstituted aliphatic chain.

25. A system to provide a nanolipoprotein particle, the systems comprising at least two of
one or more membrane-forming lipid
one or more lysolipid, and
a scaffold protein
wherein upon assembly the one or more membrane forming lipids and the scaffold protein provide the nanolipoprotein particle in which the one or more lysolipids are comprised within a membrane lipid bilayer stabilized by the scaffold protein.
